(19) United States
(12) Patent Application Publication
(10) Pub. No.: US 2024/0142467 A1
(43) Pub. Date: May 2, 2024

(54) Use of Biomarkers in the Treatment of Fibrotic Conditions with a PDE4B-inhibitor

(71) Applicant: Boehringer Ingelheim International GmbH, Ingelheim am Rhein (DE)

(72) Inventors: Christian Benedikt Otto HESSLINGER, Tettenweis (DE); Franziska Elena HERRMANN, Warthausen (DE); Yi LIU, Millwood, NY (US); Peter NICKOLAUS, Warthausen (DE)

(21) Appl. No.: 18/474,419

(22) Filed: Sep. 26, 2023

Related U.S. Application Data
(60) Provisional application No. 63/410,795, filed on Sep. 28, 2022.

(30) Foreign Application Priority Data
Dec. 9, 2022 (EP) 22 212 476.0
Sep. 4, 2023 (EP) 23 195 119.5

(51) Int. Cl. G01N 33/68; C07D 409/14

(52) U.S. Cl. CPC G01N 33/6893; C07D 409/14; G01N 2800/12; G01N 2800/52

(57) ABSTRACT

The PDE4-inhibitor of formula A′

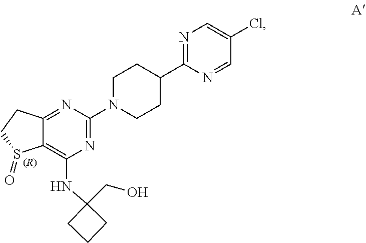

for use in a method for the treatment of a progressive fibrosing interstitial lung disease (PF-ILD), preferably IPF, in a patient comprising the following steps:
a) measuring or having measured the concentration, expression level or activity of one or more biomarkers selected from the group consisting of Krebs von den Lungen protein (KL-6), Pulmonary Surfactant Protein D (SP-D), Matrilysin (MMP7), CA-125 (also named MUC-16), CA19-9, E-selectin, sICAM-1, Stromelysin (MMP3), osteopontin (OPN), connective tissue growth factor (CTGF), Cartilage oligomeric matrix protein (COMP), prostasin, von-Willebrand-Faktor (vWF), and C-reactive protein (CRP) in the serum or plasma of a blood sample obtained from said patient,
b) comparing or having compared the concentration, expression level or activity of the one or more biomarkers as listed in step a) in said patient's blood serum or blood plasma sample to a reference concentration, expression level or activity of the respective one or more biomarkers,
c) determining or having determined that the concentration, expression level or activity of the respective one or more biomarkers as listed in step a) is modified compared to a respective reference concentration, expression or activity of that respective one or more biomarker,
d) administering to said patient a therapeutically efficient amount of the PDE4-inhibitor of formula A′.

### 1. INTRODUCTION

### 1.1 Phosphodiesterases and Their Role in Fibrosis

Phosphodiesterases (PDEs) mediate the hydrolysis of the second messengers, cyclic adenosine monophosphate (cAMP) or cyclic guanosine monophosphate. PDEs are coded by 11 gene superfamilies containing multiple genes (coding for subtypes A, B, C, etc.) that also give rise to alternative mRNA-splicing variants leading to approximately 100 PDE isoforms. PDE4 has traditionally been implicated in the regulation of inflammation and the modulation of immunocompetent cells, and the three selective PDE4 inhibitors currently available support a beneficial role for PDE4 inhibitors in inflammatory and/or autoimmune diseases (Sakkas et al., 2017, Curr. Med. Chem. 24, 3054-3067; Li et al., 2018, Front. Pharmacol. 9, 1048). The first-in-class PDE4 inhibitor, oral roflumilast (Daliresp®, Daxas®), was approved by the U.S. Food and Drug Administration in 2011 to reduce the risk of COPD exacerbations in patients with severe COPD associated with chronic bronchitis and a history of exacerbations (U.S. Food & Drug Administration, 2013, DALIRESP® (roflumilast)). Another compound, oral apremilast (Otezla®), was approved for the treatment of psoriatic arthritis and plaque psoriasis in 2014 (U.S. Food & Drug Administration, 2017, OTEZLA® (apremilast)),and for Behcet's disease in 2019. A third PDE4 inhibitor, crisaborole (Eucrisa®), was approved in 2016 for topical treatment of mild-to-moderate atopic dermatitis (U.S. Food & Drug Administration, 2016, EUCRISA™ (crisaborole)). None of these show any preferential enzymatic inhibition among the four PDE4 subtypes, A-D.

The general anti-inflammatory potential of PDE4 inhibition, as exemplified by roflumilast, is well established (Hatzelmann et al., 2010, Pulm. Pharmacol. Ther. 23, 235-256), and the use of PDE4 inhibitors in various inflammatory and immune-mediated diseases has been broadly investigated (Sakkas et al., 2017, Curr. Med. Chem. 24, 3054-3067; Li et al., 2018, Front. Pharmacol. 9, 1048). However, in the last decade, it has become increasingly clear that PDE4 may also play an important role in fibrosis, based on animal studies and on in vitro experiments evaluating the functional role of PDE4 inhibitors in fibroblasts. The attenuation of lung fibrosis by PDE4 inhibitors has been demonstrated under various experimental conditions, most widely in bleomycin-induced fibrosis in rodents. In rat models, rolipram was shown to inhibit fibrotic score, hydroxyproline content, and serum tumor necrosis factor-α (TNF-α) (Pan et al., 2009, Respirology 14, 975-982). In this initial study, the PDE4 inhibitor was administered from the beginning of bleomycin challenge, so it was not clear whether rolipram was primarily active due to inhibition of initial inflammation or inhibition of secondary fibrosis. A second early study in mice and rats, however, showed that oral roflumilast was active both in preventive and in therapeutic protocols in a dose-dependent manner (Cortijo et al., 2009, Br. J. Pharmacol. 156, 534-544). In lung extracts, roflumilast inhibited histologically assessed fibrosis, hydroxyproline content, and the mRNA expression of TNF-α, transforming growth factor-β (TGF-β), connective tissue growth factor (CTGF), α1 collagen, endothelin-1, and mucin 5ac. In bronchoalveolar lavage fluid (BALF), the levels of TNF-α, interleukin (IL)-13, TGF-β, and mucin 5ac, the formation of lipid hydroperoxides, and the influx of inflammatory cells (e.g. neutrophils and macrophages) were inhibited. Besides fibrosis, right ventricular hypertrophy and vascular remodeling (pulmonary arteries) were positively influenced by roflumilast. The same group later on also demonstrated that the metabolome associated with pulmonary fibrosis in bleomycin mice was modulated by roflumilast. Levels of the amino acids (AAs) glycine and proline, involved in collagen formation/structure, were lowered by roflumilast, while lung glutathione and plasma tetrahydrobiopterin were increased, suggesting an alteration in oxidative equilibrium by roflumilast (Milara et al., 2015, PLoS One 10, e0133453). Another PDE4 inhibitor, cilomilast, was shown to inhibit late-stage lung fibrosis and tended to reduce collagen content in bleomycin mice, although no effect on TGF-β1 and collagen type (Col) 1A1 expression was found (Udalov et al., 2010, BMC Pulm. Med. 10, 26).

Improvement of lung fibrosis by PDE4 inhibition was not limited to the bleomycin model. In a murine model of lung fibrosis targeting type II alveolar epithelial cells in transgenic mice expressing the diphtheria toxin receptor under the control of the murine surfactant protein C promoter, roflumilast lowered lung hydroxyproline content and mRNA expression of TNF-α, fibronectin (FN), and CTGF (Sisson et al., 2018, Physiol. Rep. 6, e13753). Interestingly, roflumilast was active both in a preventive and in a therapeutic regimen, and under the latter conditions appeared to be therapeutically equipotent to pirfenidone and nintedanib. Furthermore, in a mouse model of chronic graft-versus-host disease, lung fibrosis was attenuated by oral roflumilast (Kim et al., 2016, Exp. Hematol. 44, 332-341.e334). Roflumilast inhibited fibrosis, collagen deposition, hydroxyproline and TGF-β1 content, cell infiltration, and expression of mRNA for IL-6 and IL-1β. In addition, in BALF inflammatory cells (macrophages, lymphocytes, neutrophils, and eosinophils), expression of mRNA for IL-6, IL-1β, and monocyte chemotactic protein-1 was inhibited by roflumilast. In a rabbit tuberculosis model, pulmonary damage and fibrosis were shown to be inhibited by two PDE4 inhibitors from Celgene, CC-3052 (Subbian et al., 2011, Am. J. Pathol. 179, 289-301) and CC-11050 (Subbian et al., 2016, EBioMedicine 4, 104-114). PDE4 inhibition improved antibiotic therapy and lung fibrosis by positively influencing collagen deposition and mRNA expression of various matrix metalloproteinases, including matrix metalloproteinase 12.

Besides the lung, beneficial effects of PDE4 inhibition on fibrosis have been demonstrated in several other organs including skin, liver, kidney, and colon. For example, in various preclinical mouse models of SSc, skin fibrosis induced by either bleomycin or topoisomerase I and chronic graft-versus-host disease was inhibited by rolipram and apremilast (Maier et al., 2017, Ann. Rheum. Dis. 76, 1133-1141). This group did not find direct inhibitory effects of PDE4 inhibition on the release of profibrotic cytokines (IL-6, IL-13, TGF-β1/β2) in fibroblasts and M2 macrophages purified from peripheral blood of patients with SSc, which may be due to the lack of an exogenous cAMP trigger under the experimental conditions used. In a model of unilateral ureteral obstruction-induced obstructive nephropathy in mice, rolipram was shown to inhibit renal interstitial fibrosis (Ding et al., 2017, Antioxid. Redox Signal. 29, 637-652). In mouse primary tubular epithelial cells in vitro, TGF-β up-regulated PDE4A/B, and rolipram inhibited TGF-β-induced damage, FN expression, and deficiency of mitochondrial biogenesis. roflumilast inhibited diethylnitrosamine-induced liver fibrosis, hydroxyproline deposition, and TGF-β1 expression in rats (Essam et al., 2019, Life Sci. 222, 245-254). Similarly, rolipram inhibited collagen deposition, α-smooth muscle actin (α-SMA) staining, and mRNA expression, as well as the expression of TGF-β1 mRNA and TNF-α protein, in a bile duct ligation-induced hepatic fibrosis model in rats, with up-regulation of PDE4A, B, and D (Gobejishvili 2019). In hepatic stellate cells in vitro, rolipram inhibited mRNA expression of α-SMA and Col1A2 (Gobejishvili et al., 2013, J. Pharmacol. Exp. Ther. 347, 80-90). With respect to colonic tissue, rolipram inhibited collagen and TGF-β1 in a model of trinitrobenzene sulfonic acid-induced colitis in rats (Videla et al., 2006, J. Pharmacol. Exp. Ther. 316, 940-945), and apremilast inhibited fibrosis in colon, collagen deposition, and the expression of genes related to fibrosis in a model of dextran sulfate sodium-induced colitis ulcerosa in mice (Li et al., 2019, Br. J. Pharmacol. 176, 2209-2226). In a murine cecal abrasion model, rolipram inhibited fibrotic reactions, indicating that PDE4 inhibition has the potential to prevent postoperative intra-abdominal adhesions (Eser et al., 2012, Dis. Colon Rectum 55, 345-350). Adhesions are assumed to result from laparotomy by abnormal healing. In support of this assumption, rolipram has been shown to be active in a subcutaneous or intraperitoneal polyether-polyurethane sponge implant model in mice by inhibiting intra-implant collagen and TGF-β1 deposition (Mendes et al., 2009, Microvasc. Res. 78, 265-271). Thus, in various animal models, the beneficial impact of selective PDE4 inhibition on fibrosis has been proven, most extensively in the lung but also in several other organs. While the specific target(s) of PDE4 inhibitors in fibrotic diseases are largely unknown, it is tempting to speculate that they act either indirectly via inhibition of pro-inflammatory cells and mediators, and/or directly by inhibiting the typical effector cells (fibroblasts, myofibroblasts) mediating fibrosis.

### 1.2 Progressive Fibrosing Interstitial Lung Diseases (PF-ILD)

Interstitial lung diseases (ILDs) comprise a heterogeneous group of lung diseases affecting the interstitium, distinct from obstructive airway diseases such as asthma or chronic obstructive pulmonary disease (COPD). Prolonged ILD may result in pulmonary fibrosis, but this is not always the case. The most extensively studied ILD is idiopathic pulmonary fibrosis (IPF), which is characterized by progressive pulmonary fibrosis. Non-IPF ILDs may include connective tissue disease-related ILDs such as those related to rheumatoid arthritis and other autoimmune diseases, systemic sclerosis associated ILD (SSc-ILD), and polymyositis/dermatomyositis, and ILDs related to chronic sarcoidosis, chronic hypersensitivity pneumonitis, idiopathic non-specific interstitial pneumonia, and exposure-related diseases such as asbestosis and silicosis (Cottin et al, Eur. Respir. Rev. 28, 180100; Kolb, M., and Vasakova, M. (2019), Respir. Res. 20, 57). Up to 40% of patients with these ILDs may develop a progressing fibrotic phenotype.

Progressive fibrosing ILDs are associated with high mortality, with median post-diagnosis survival in patients with IPF estimated at 2-5 years (Raghu, G., Chen, S. Y., Yeh, W. S., Maroni, B., Li, Q., Lee, Y. C., and Collard, H. R. (2014). Idiopathic pulmonary fibrosis in US Medicare beneficiaries aged 65 years and older: incidence, prevalence, and survival, 2001-11. Lancet Respir. Med. 2, 566-572). Progression of fibrosing ILD is reflected in various parameters, including decline in pulmonary function, decrease in exercise capacity, deterioration in quality of life, worsening of cough and dyspnea, acute exacerbations, and increase of morphologic abnormalities (Cottin et al. Eur. Respir. Rev. 28, 180100, 2019; Kolb and Vasakova, 2019, Respir. Res. 20, 57). In patients with IPF, forced vital capacity (FVC) is a well-established predictor of mortality, and acute exacerbations are associated with very high mortality. Although corticosteroids and/or immunosuppressive drugs are sometimes used off-label to treat progressive fibrosing ILDs, currently the only approved treatments to slow disease progression in IPF are nintedanib and pirfenidone (Richeldi et al., 2018, Eur. Respir. Rev. 27, 180074). Nintedanib has been approved in the US since 2014 (U.S. Food & Drug Administration, 2020, OFEV® (nintedanib)), and in Europe and Japan since 2015 (European Medicines Agency, 2021b, OFEV® (nintedanib)), while pirfenidone was approved in Japan in 2008, in Europe in 2011 (European Medicines Agency, 2021a, Esbriet (pirfenidone)), and in the US in 2014 (U.S. Food & Drug Administration, 2019, ESBRIET® (pirfenidone)). Lung transplantation is the only potentially curative treatment for IPF and the medical need in IPF and other progressive fibrosing ILDs remains high.

However, in patients with IPF having a mild or moderate impairment of FVC (≥50% predicted), both presently approved medications pirfenidone and nintedanib, can only reduce the decline in FVC, consistent with a slowing of disease progression, but both are not able to stop or even reverse or heal the symptoms of IPF (Tzouvelekis et al Ther. Clin. Risk Management 2015, 11, 359-370).

Nevertheless, both treatment options, either with pirfenidone or with nintedanib, show significant beneficial effects in slowing down IPF disease progression.

The most prominent side effects associated with both, nintedanib and pirfenidone, are gastrointestinal events, particularly diarrhea, nausea, vomiting, abdominal pain, decreased appetite and a decreased body weight. In case that gastrointestinal side effects arise, they are usually managed either by treatment interruption, dose reduction or symptomatic treatment of the gastrointestinal side effects (see Mazzei et al, Ther. Adv. Respir. Dis. 2015, Vol. 9 [3], pp. 121-129).

Due to these “accumulative gastrointestinal side effects” of pirfenidone on the one hand and of nintedanib on the other hand a combination treatment for IPF by a combination of pirfenidone and nintedanib is not frequently used. Investigations have shown that a combination treatment with pirfenidone and nintedanib leads to increased gastrointestinal side effects, in particular to diarrhoea, nausea, vomiting, and upper abdominal pain (Vancheri et al., nintedanib with Add-on pirfenidone in Idiopathic Pulmonary Fibrosis: Results of the INJOURNEY Trial. Am J Respir Crit Care Med. 2018, Feb. 1; 197 (3): 356-363).

Consequently, due to the fact that both active agents which are so far approved for the treatment for IPF, pirfenidone and nintedanib, are—when administered alone—not able to stop or to heal IPF, but instead can only slow down the IPF disease progression to a certain percentage (Tzouvelekis et al Ther. Clin. Risk Management 2015, 11, 359-370), and due to the fact that additionally both nintedanib and pirfenidone show significant gastrointestinal side effects which accumulate when both compounds are combined, there is still a significant medical need for improved methods of treatment of IPF/PF-ILD.

### 1.3 Biomarkers in PF-ILD and in IPF

A biomarker is defined as an indicator of normal biological processes, pathogenic processes (e.g. prognostic biomarkers), or responses to an exposure or intervention, including therapeutic interventions (e.g. pharmacodynamic biomarkers, outcome-related biomarkers). Sources of biomarkers that may inform diagnosis, outcomes and treatment response in PF-ILD include the peripheral blood, airway and lung parenchyma. Peripheral blood is easily obtained, and acquisition requires little training beyond phlebotomy. As described in Bowman et al, Front. Med. 8: 680997, doi: 10.3389/fmed.2021.680997 many diagnostic interstitial lung disease (ILD) biomarkers such as CA 19-9, CA-125, sICAM-1 etc discriminate different subtypes of ILD versus non-ILD controls. Furthermore, Stainer et al, Int. J. Mol. Sci. 2021, 22, 6255 focusses more on idiopathic pulmonary fibrosis (IPF) and shows evidence that IPF has different clinical phenotypes which are characterized by a variable disease course over time. Therefore, diagnostic, prognostic and theranostic biomarkers (outcome-related or pharmacodynamic biomarkers) could become useful tools helping facilitate IPF diagnosis, monitoring IPF disease progression and treatment efficacy.

### 1.4 Prior Art

In addition to the approved PDE4 inhibitors roflumilast and apremilast—many further patent applications drawn on other PDE4-inhibitors with improved properties have been published:
Pteridines as PDE4-inhibitors in WO 2006/056607, WO 2006/058869, WO 2006/058868 and WO 2006/058867.
Piperazino-Dihydrothienopyrimidines as PDE4-inhibitors in WO 2006/111549, WO 2007/118793 and WO 2009/050242.
Piperidino-Dihydrothienopyrimidines as PDE4-inhibitors in WO 2009/050248 and in WO 2013/026797.

The PDE4-inhibitor of formula A

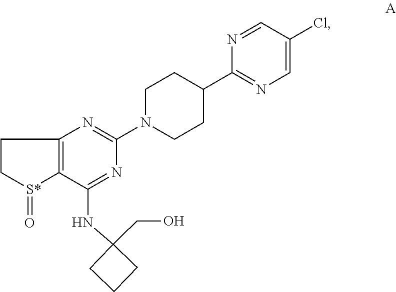

wherein the S* is a sulphur atom that represents a chiral center,
and in particular the PDE4-inhibitor of formula A′

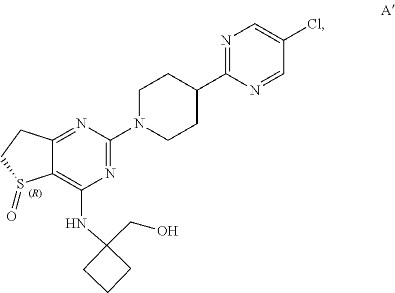

wherein the (R) at the sulphur atom represents the chiral center at the sulphur atom being in the R-configuration,
has been disclosed in WO 2013/026797 and was proposed as a new therapeutic option in diverse diseases including IPF. It had been shown that the PDE4-inhibitor of formulas A inhibits preferentially the PDE4 B subtype.

WO2019/081235 discloses a combination of nintedanib and of the PDE4-inhibitor of formula A—and in particular of formula A′—for the treatment of PF-ILD, preferably IPF, which shows in an in vitro assay using human lung fibroblasts a synergistic over-additive effect with respect to fibroblast proliferation.

EP21218202.6 discloses new pharmaceutical compositions of the PDE4-inhibitor of formula A—and in particular of formula A′—combined with a new dose regimen.

EP21218207.5 discloses new combinations of the PDE4-inhibitor of formula A—and in particular of formula A′.

However, none of the above-mentioned pieces of prior art disclose any fibrosis-related biomarker with a level, concentration or expression that may be influenced by the treatment with the PDE4-inhibitor of formula A′ (=“biomarkers with pharmacodynamic potential”) or that are “outcome-related” (which means that these biomarkers vary in dependency of the therapeutic outcome/efficacy of the PDE4-inhibitor of formula A′).

Regarding fibrosis-related biomarkers it is known from Zhang et al, Curr Opin Pulm Med 212; 18 (5): 441-446 that high blood concentrations of KL-6 (also known as MUC1) have been shown to be predictive of decreased survival in IPF and that high blood plasma concentrations of MMP-7, sICAM-1 and IL-8 were predictive of poor overall survival in IPF-patients.

In Stainer et al, Int J Mol Sci 2021, 22, 6255 it is described that different variants of surfactant proteins in serum, such as SP-A and SP-D had been identified as diagnostic markers in IPF: for instance serum levels of SP-D in IPF-patients and in other non-IPF-ILDs had been higher than those in healthy controls and further KL-6 was increased in blood serum of patients suffering from several ILDs including IPF.

However, neither in Zhang et al, Curr Opin Pulm Med 212; 18 (5): 441-446 nor in Stainer et al, Int J Mol Sci 2021, 22, 6255 it is described whether and/or—to which extend—certain standard of care IPF therapeutics such as for example nintedanib or pirfenidone influence the blood plasma or serum concentrations of the above-mentioned biomarkers or whether the therapeutic outcome of nintedanib or pirfenidone treatments options influence certain biomarker levels. In particular, the possible influence of new potential IPF therapeutics such as for example the PDE4-inhibitor of formula A′ on fibrosis-related biomarkers was not mentioned or discussed in the above-mentioned articles Zhang et al and Stainer et al. and therefore it is completely unknown whether certain known fibrosis-related biomarkers show a “pharmacodynamic potential” for new IPF- or PF-ILD-medications such as the PDE4-inhibitor of formula A′ or not. Further, the above-mentioned prior art documents do not state whether specific fibrosis-related biomarkers show an “outcome-related potential” during the treatment with specific new PF-ILD- or IPF-medications such as for example the PDE4-inhibitor of formula A′.

In the phase 2 study NCT04419506 the PDE4-inhibitor of formula A′ was tested against Placebo in IPF patients without antifibrotic background treatment (“non-AF background”) and in IPF patients with antifibrotic background treatment selected from either nintedanib or pirfenidon (“AF-background”). In both IPF patient groups—in the “non-AF-background” group and in the “AF-background” group—those patients that had been treated with 18 mg of the PDE4-inhibitor of formula A′ twice daily showed a significantly slowed decrease in lung function (“Forced Vital Capacity” (FVC)) compared to patients that had obtained Placebo.

The patients of the phase 2 study were further analyzed for the concentration/level of certain fibrosis-related biomarkers and other biomarkers in their blood plasma or blood serum.

The results of these analyses as described in detail in Chapter 3.2.3.2 of this application show that in the “non-AF-group” the blood concentrations/levels of the biomarkers KL-6, SP-D, CA-125 (MUC-16 or MUCIN-16), CA19-9, Matrilysin (=MMP7), COMP, Prostasin, E-selectin and vWF are decreased over time in IPF patients which had been treated with the PDE4-inhibitor of formula A′ compared to IPF patients that had obtained Placebo (see Table 3 and FIG. 6, 8, 10, 14, 18, 20, 22, 24, 28). Further, these decreased blood concentrations/levels of the aforementioned biomarkers in IPF patients treated with the compound of formula A′ simultaneously occured with a significantly slowed decrease in lung function compared to IPF patients that had obtained Placebo (see FIG. 1).

Additionally it could be shown that in the “non-AF-group” the blood concentration/level of the fibrosis-related biomarker C-reactive protein (CRP) is increased over time in IPF patients that had been treated with the PDE4-inhibitor of formula A′ compared to IPF patients which had obtained Placebo instead (see Table 3 and FIG. 26). Further, this increased blood concentration/level of CRP in IPF patients treated with the compound of formula A′ simultaneously occured with a significantly slowed decrease in lung function compared to IPF patients that had obtained Placebo (see FIG. 1).

These observations could imply that so far untreated IPF patients which exhibit a biomarker selected from the group consisting of KL-6, SP-D, CA-125, CA19-9, Matrilysin (MMP-7), COMP, Prostasin, E-selectin and vWF in their blood serum or plasma in a high concentration/level that exceeds a certain reference concentration/threshold level could particularly profit from the treatment with the PDE4-inhibitor of formula A′.

Further, these observations could imply that so far untreated IPF patients which exhibit low concentrations/levels of CRP in their blood serum or plasma—in particular when a certain reference concentration/level is not reached—could particularly profit from the treatment with the PDE4-inhibitor of formula A′.

Additionally the results of the analyses as described in Chapter 3.2.3.2 of this application show that in the “AF-group” (the group that had obtained background antifibrotic treatment with either nintedanib or pirfenidone) the blood concentrations/levels of the biomarkers KL-6, SP-D, Matrilysin (=MMP7) and E-selectin of those IPF patients that had been treated with the PDE4-inhibitor of formula A′ were decreased over time compared to those IPF-patients that had obtained Placebo instead (see Table 4 and FIG. 7, 9, 15, 29).

Further, it could be shown that in the “AF-group” the blood concentration/level of the fibrosis-related biomarker C-reactive protein (CRP) in IPF patients that had been treated with the PDE4-inhibitor of formula A′ was increased over time compared to IPF patients that had obtained Placebo instead (see Table 4 and FIG. 27).

These observations could imply that IPF patients that already received antifibrotic background treatment (with either nintedanib, pirfenidone or any pharmaceutically acceptable salt thereof) who exhibit a biomarker selected from the group consisting of KL-6, SP-D, Matrilysin (MMP-7) and E-selectin in their blood serum or plasma -in particular when a certain threshold/reference concentration/level/activity is exceeded- could particularly profit from the treatment with the PDE4-inhibitor of formula A′ (either alone or in combination with antifibrotic background treatment selected from either nintedanib or pirfenidone).

Further, these observations could imply that IPF patients that already received antifibrotic background treatment (with either nintedanib, pirfenidone or any pharmaceutically acceptable salt thereof) who exhibit low concentrations/levels of CRP in their blood serum or plasma, in particular when a certain threshold/reference concentration/level/activity is not reached, could particularly profit from the treatment with the PDE4-inhibitor of formula A′ (either alone or in combination with antifibrotic background treatment selected from either nintedanib or pirfenidone).

FIG. 30 shows that in particular the fibrosis-related biomarkers KL-6, SP-D and MMP-7 (Matrilysin) are decreased during the time of treatment with the PDE4-inhibitor of formula A′ and therefore show a “pharmacodynamic potential” with respect to the treatment with the PDE4-inhibitor of formula A′ in IPF patients (regardless whether the patients had additionally been subjected to antifibrotic background treatment or not). Additionally, FIG. 30 shows that C-reactive protein (CRP) is increased during the time of treatment with the PDE4-inhibitor of formula A′ and therefore shows a “pharmacodynamic potential” with respect to the treatment with the PDE4-inhibitor of formula A′ in IPF patients (regardless whether the patients had additionally been subjected to antifibrotic background treatment or not).

FIG. 31 shows that in particular baseline levels of KL-6, SP-D, CA-125 (=Mucin-16), CA19-9 and sICAM-1 are negatively associated with a decrease-from-baseline in lung function, measured as change-from-baseline (CfB) in FVC and as change-from-baseline (CfB) in Dlco % pred. at week 12, in IPF patients of the Placebo-groups only. Therefore, FIG. 31 shows that in particular KL-6, SP-D, CA-125 (=Mucin-16), CA19-9 and sICAM-1 show a “prognostic potential” for the progression of IPF.

FIG. 32 further shows that in IPF patients during the treatment with the PDE4-inhibitor of formula A′ in particular the change in levels of biomarkers KL-6, SP-D, E-selectin and sICAM-1 shows an association with the Change from Baseline (CfB) of the Forced Vital Capacity (FVC) and with the Change from Baseline (CfB) in the Diffusing capacity or Transfer factor of the lung carbon monoxide (Dlco) at week 12, in both, the “non-AF group” and the “AF group”. FIG. 32 therefore shows that in particular the biomarkers KL-6, SP-D, E-selectin and sICAM-1 have an“outcome-related potential” during the treatment with the PDE4-inhibitor of formula A′.

One can conclude from these biomarker analyses of this phase 2 trial for the PDE4-inhibitor of formula A′ that particularly those biomarkers having a “pharmacodynamic potential”, that means KL-6, SP-D and MMP-7, as well as those biomarkers having a “prognostic potential”, that means KL-6, SP-D, CA-125, CA19-9 and sICAM-1, and those biomarkers showing a potential link with the “outcome” (FVC and Dlco % pred at week 12), that means KL-6, SP-D, MMP-7, E-selectin and sICAM-1 are preferably suitable to be used to identify IPF patients that could extraordinarily profit from a treatment with the PDE4-inhibitor of formula A′ or are preferably suitable to be used to monitor treatment progress with the PDE4-inhibitor of formula A′.

Since KL-6 and SP-D are biomarkers which combine all three, a “pharmacodynamic potential” for the treatment with the PDE4-inhibitor of formula A′, a potential link with the “outcome” or treatment success (FVC and Dlco % at week 12 of the treatment with the PDE4-inhibitor of formula A′) and additionally a “prognostic potential” for the progression of the IPF (see FIG. 33), biomarkers KL-6 and SP-D are particularly suitable to be used to identify IPF-patients that could extraordinarily profit from a treatment with the PDE4-inhibitor of formula A′ or are particularly suitable to be used to monitor treatment progress with the PDE4-inhibitor of formula A′.

### 2. DESCRIPTION OF THE INVENTION

In a first aspect the invention concerns the PDE4-inhibitor of formula A′

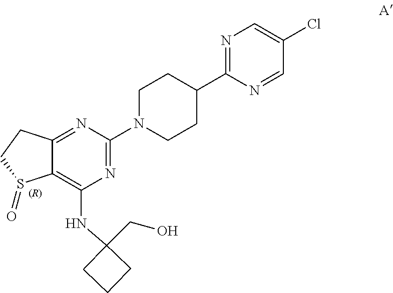

for use in a method for treating a progressive fibrosing interstitial lung disease (PF-ILD) in a patient exhibiting one or more biomarkers selected from the group consisting of KL-6, SP-D, MMP7, CA-125, CA19-9, E-selectin, sICAM-1, MMP3, OPN, CTGF, COMP, prostasin, vWF, and CRP.

In a preferred embodiment the progressive fibrosing interstitial lung disease (PF-ILD) is idiopathic pulmonary fibrosis (IPF).

In a further preferred embodiment the PDE4-inhibitor of formula A′ is for use in a method for treating a progressive fibrosing interstitial lung disease (PF-ILD) in a patient exhibiting one or more biomarkers selected from the group consisting of KL-6, SP-D, MMP7, OPN, CA-125, CA19-9, sICAM-1 and E-selectin.

In a further preferred embodiment the PDE4-inhibitor of formula A′ is for use in a method for treating a progressive fibrosing interstitial lung disease (PF-ILD) in a patient exhibiting one or more biomarkers selected from the group consisting of KL-6, SP-D, MMP7, and E-selectin.

In a further preferred embodiment the PDE4-inhibitor of formula A′ is for use in a method for treating a progressive fibrosing interstitial lung disease (PF-ILD) in a patient exhibiting one or more biomarkers selected from the group consisting of KL-6, SP-D and MMP7.

In a particularly preferred embodiment the PDE4-inhibitor of formula A′ is for use in a method for treating a progressive fibrosing interstitial lung disease (PF-ILD) in a patient exhibiting one or more biomarkers selected from the group consisting of KL-6 and SP-D,

In another preferred embodiment the PDE4-inhibitor of formula A′ is for use in a method for treating a progressive fibrosing interstitial lung disease (PF-ILD) in a patient exhibiting one or more biomarkers selected from the group consisting of COMP, vWF and prostasin.

In a particularly preferred embodiment the PDE4-inhibitor of formula A′ is for use in a method for treating a progressive fibrosing interstitial lung disease (PF-ILD) in a patient exhibiting KL-6.

In another particularly preferred embodiment the PDE4-inhibitor of formula A′ is for use in a method for treating a progressive fibrosing interstitial lung disease (PF-ILD) in a patient exhibiting SP-D.

In a second aspect the invention concerns a method for the treatment of a progressive fibrosing interstitial lung disease (PF-ILD), preferably idiopathic pulmonary fibrosis (IPF), in a patient comprising administering to the patient a pharmaceutically effective amount of the PDE4-inhibitor of formula A′

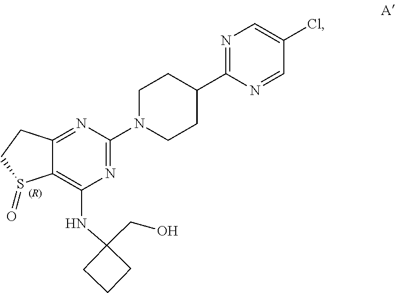

wherein the patient exhibits one or more biomarkers selected from the group consisting of KL-6, SP-D, MMP7, CA-125, CA19-9, OPN, CTGF, E-selectin, sICAM-1, MMP3, COMP, prostasin, vWF and CRP.

In a preferred embodiment the PDE4-inhibitor of formula A′ is administered to the patient in the dose of 18 mg twice daily.

In a further preferred embodiment the patient exhibits one or more biomarkers selected from the group consisting of KL-6, SP-D, CA-125, CA19-9, MMP7, OPN, sICAM-1 and E-selectin.

In a further preferred embodiment the patient exhibits one or more biomarkers selected from the group consisting of KL-6, SP-D, MMP7 and E-selectin.

In a further preferred embodiment the patient exhibits one or more biomarkers selected from the group consisting of KL-6, SP-D and MMP7.

In a further preferred embodiment the patient exhibits one or more biomarkers selected from the group consisting of KL-6 and SP-D.

In a particularly preferred embodiment the patient exhibits KL-6.

In another particularly preferred embodiment the patient exhibits SP-D.

In another preferred embodiment the patient exhibits one or more biomarkers selected from the group consisting of COMP, vWF and prostasin.

In a third aspect, the invention refers to the PDE4-inhibitor of formula A′

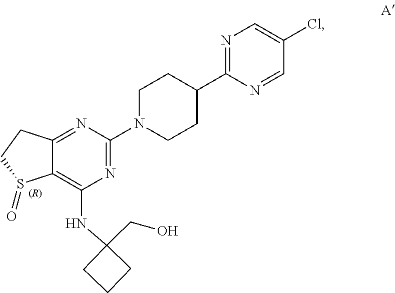

for use in a method for treating a progressive fibrosing interstitial lung disease (PF-ILD) in a patient who has been determined to exhibit one or more biomarkers selected from the group consisting of KL-6, SP-D, MMP7, CA-125, CA19-9, OPN, CTGF, E-selectin, sICAM-1, MMP3, COMP, prostasin, vWF and CRP.

In a preferred embodiment the progressive fibrosing interstitial lung disease (PF-ILD) is idiopathic pulmonary fibrosis (IPF).

In a preferred embodiment the PDE4-inhibitor of formula A′ is administered to the patient in the dose of 18 mg twice daily.

In a further preferred embodiment the patient exhibits one or more biomarkers selected from the group consisting of KL-6, SP-D, CA-125, CA19-9, MMP7, OPN, sICAM-1 and E-selectin.

In a further preferred embodiment the patient exhibits one or more biomarkers selected from the group consisting of KL-6, SP-D, MMP7 and E-selectin.

In a further preferred embodiment the patient exhibits one or more biomarkers selected from the group consisting of KL-6, SP-D and MMP7.

In a particularly preferred embodiment the patient exhibits one or more biomarkers selected from the group consisting of KL-6 and SP-D.

In a particularly preferred embodiment the patient exhibits biomarker KL-6.

In another particularly preferred embodiment the patient exhibits biomarker SP-D.

In another preferred embodiment the patient exhibits one or more biomarkers selected from the group consisting of COMP, vWF and prostasin.

In a fourth aspect, the invention refers to a method for the treatment of a progressive fibrosing interstitial lung disease (PF-ILD) in a patient comprising
Determining that the patient exhibits one or more biomarkers selected from the group consisting of KL-6, SP-D, MMP7, CA-125, CA19-9, OPN, CTGF, E-selectin, sICAM-1, MMP3, COMP, prostasin, vWF and CRP, and
Administering to the patient a pharmaceutically effective amount of the PDE4-inhibitor of formula A′

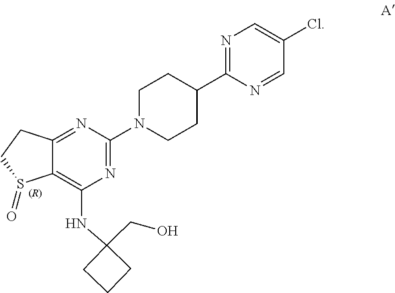

In a preferred embodiment the progressive fibrosing interstitial lung disease (PF-ILD) is idiopathic pulmonary fibrosis (IPF).

In a preferred embodiment the PDE4-inhibitor of formula A′ is administered to the patient in the dose of 18 mg twice daily.

In a further preferred embodiment the patient exhibits one or more biomarkers selected from the group consisting of KL-6, SP-D, CA-125, CA19-9, MMP7, OPN, sICAM-1 and E-selectin.

In a further preferred embodiment the patient exhibits one or more biomarkers selected from the group consisting of KL-6, SP-D, MMP7 and E-selectin.

In a further preferred embodiment the patient exhibits one or more biomarkers selected from the group consisting of KL-6, SP-D and MMP7.

In a particularly preferred embodiment the patient exhibits one or more biomarkers selected from the group consisting of KL-6 and SP-D.

In a particularly preferred embodiment the patients exhibits biomarker KL-6.

In another particularly preferred embodiment the patients exhibits biomarker SP-D.

In another preferred embodiment the patient exhibits one or more biomarkers selected from the group consisting of COMP, vWF and prostasin.

In a fifth aspect, the inventions concerns the PDE4-inhibitor of formula A′

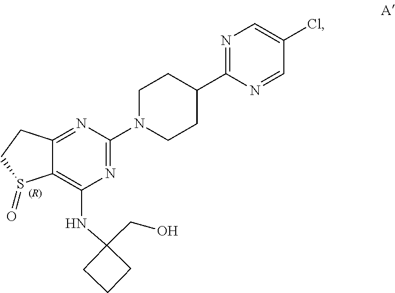

for use in a method for the treatment of a progressive fibrosing interstitial lung disease (PF-ILD) in a patient comprising the following steps:
a) measuring or having measured the concentration, expression level or activity of one or more biomarkers selected from the group consisting of Krebs von den Lungen protein (KL-6), Pulmonary Surfactant Protein D (SP-D), Matrilysin (MMP7), CA-125 (also named MUC-16), CA19-9, E-selectin, Stromelysin (MMP3), osteopontin (OPN), connective tissue growth factor (CTGF), Cartilage oligomeric matrix protein (COMP), prostasin, von-Willebrand-Factor (vWF), soluble Intercellular Adhesion Molecule 1 (sICAM1) and C-reactive protein (CRP) in the serum or plasma of a blood sample obtained from said patient,
b) comparing or having compared the concentration, expression level or activity of the one or more biomarkers as listed in step a) in said patient's blood serum or blood plasma sample to a reference concentration, expression level or activity of the respective one or more biomarkers,
c) determining or having determined that the concentration, expression level or activity of the respective one or more biomarkers as listed in step a) is modified compared to a respective reference concentration, expression or activity of that respective one or more biomarker,
d) administering to said patient a therapeutically efficient amount of the PDE4-inhibitor of formula A′.

Preferably the one or more biomarkers of step a) are selected from the group consisting of KL-6, SP-D, MMP7, MMP3, OPN, CTGF, COMP, prostasin, vWF, CA-125, CA19-9, sICAM-1 and E-selectin and then step c) comprises determining or having determined that the concentration, expression level or activity of the respective one or more biomarkers selected from the group consisting of KL-6, SP-D, MMP7, MMP3, OPN, CTGF, COMP, prostasin, vWF, CA-125, CA19-9, sICAM-1 and E-selectin is increased compared to a respective reference concentration, expression or activity of that respective one or more biomarker.

More preferred the one or more biomarkers of step a) are selected from the group consisting of KL-6, SP-D, MMP7, CA-125, CA19-9, OPN, sICAM-1 and E-selectin and then step c) comprises determining or having determined that the concentration, expression level or activity of the respective one or more biomarkers selected from the group consisting of KL-6, SP-D, MMP7, CA-125, CA19-9, OPN, sICAM-1 and E-selectin is increased compared to a respective reference concentration, expression or activity of that respective one or more biomarker.

More preferred the one or more biomarkers of step a) are selected from the group consisting of KL-6, SP-D, MMP7 and E-selectin and then step c) comprises determining or having determined that the concentration, expression level or activity of the respective one or more biomarkers selected from the group consisting of KL-6, SP-D, MMP7 and E-selectin is increased compared to a respective reference concentration, expression or activity of that respective one or more biomarker.

More preferred the one or more biomarkers of step a) are selected from the group consisting of KL-6, SP-D and MMP7 and then step c) comprises determining or having determined that the concentration, expression level or activity of the respective one or more biomarkers selected from the group consisting of KL-6, SP-D and MMP7 is increased compared to a respective reference concentration, expression or activity of that respective one or more biomarker.

Most preferred the one or more biomarkers of step a) are selected from the group consisting of KL-6 and SP-D, and then step c) comprises determining or having determined that the concentration, expression level or activity of the respective one or more biomarkers selected from the group consisting of KL-6 and SP-D is increased compared to a respective reference concentration, expression or activity of that respective one or more biomarker.

Particularly preferred the one or more biomarkers of step a) is KL-6.

Particularly preferred the one or more biomarkers of step a) is MMP-7.

Particularly preferred the one or more biomarkers of step a) is SP-D.

In particular the one or more biomarkers of step a) is KL-6 and then the reference activity of KL-6 in the blood serum of the patients in step c) is >1000 U/ml

In another preferred embodiment, the one or more biomarkers of step a) is C-reactive protein (CRP) and then step c) comprises determining or having determined that the concentration of CRP is decreased compared to a respective reference concentration of CRP.

The progressive fibrosing interstitial lung disease (PF-ILD) to be treated is preferably idiopathic pulmonary fibrosis (IPF).

In one preferred embodiment the patient has already been treated by an antifibrotic compound selected from nintedanib, pirfenidone or any pharmaceutically acceptable salts thereof prior to steps a), b), c) and d) and the treatment with said antifibrotic compound is continued as background treatment during the treatment with the compound of formula A′ in step d).

In another preferred embodiment the patient has already been treated by an antifibrotic compound selected from nintedanib, pirfenidone or any pharmaceutically acceptable salts thereof prior to steps a), b), c) and d) and the treatment with said antifibrotic compound is not continued as background treatment during the treatment with the compound of formula A′ in step d).

In a further preferred embodiment 18 mg of the PDE4-inhibitor of formula A′ is administered twice daily to the patient in step d).

In another preferred embodiment the concentration, expression level or activity of the respective one or more biomarker in step c) is a concentration, expression level or activity of the respective one or more biomarker that is larger than the reference concentration and that is considered as prognostic for PF-ILD-progression (or preferably for IPF-progression).

In a sixth aspect, the invention concerns the use of one or more biomarkers selected from the group consisting of KL-6, SP-D, MMP7, MMP3, OPN, CTGF, COMP, prostasin, vWF, CA-125, CA19-9, CRP and E-selectin in a method of treating a PF-ILD—preferably IPF—by administering a pharmaceutically effective amount of the PDE4-inhibitor of formula A′.

In a preferred embodiment the one or more biomarkers as mentioned above are selected from the group consisting of KL-6, SP-D, MMP7, CA-125, CA19-9, OPN, sICAM-1 and E-selectin.

In a more preferred embodiment the one or more biomarkers as mentioned above are selected from the group consisting of KL-6, SP-D, MMP7 and E-selectin.

In a more preferred embodiment the one or more biomarkers as mentioned above are selected from the group consisting of KL-6, SP-D and MMP7.

In a more preferred embodiment the one or more biomarkers as mentioned above are selected from the group consisting of KL-6 and SP-D.

In a particularly preferred embodiment the one or more biomarkers is KL-6.

In another particularly preferred embodiment the one or more biomarkers is SP-D.

In a preferred embodiment the one or more biomarkers as mentioned above are selected from the group consisting of COMP, prostasin and vWF.

In another preferred embodiment the pharmaceutically effective amount of the PDE4-inhibitor of formula A′ is 18 mg twice daily.

In a seventh aspect, the invention concerns a method of monitoring the treatment success in PF-ILD-patients, preferably in IPF-patients, who had been treated with a PDE4-inhibitor of formula A′

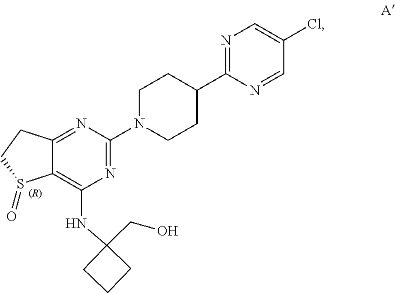

comprising the steps
a) measuring or having measured the concentration, expression level or activity of one or more biomarkers selected from the group consisting of Krebs von den Lungen protein (KL-6), Pulmonary Surfactant Protein D (SP-D), Matrilysin (MMP7), CA-125 (also named MUC-16), CA19-9, E-selectin, soluable Intercellular adhesion molecule 1 (sICAM1), Stromelysin (MMP3), osteopontin (OPN), connective tissue growth factor (CTGF), Cartilage oligomeric matrix protein (COMP), prostasin, von-Willebrand-Faktor (vWF), and C-reactive protein (CRP) in the serum or plasma of a blood sample obtained from said patient,
b) comparing or having compared the measured concentration, expression level or activity of the one or more biomarkers as listed in step a) in said patient's blood serum or blood plasma sample to a reference concentration, expression level or activity of the respective one or more biomarkers
or
comparing or having compared the concentration, expression level or activity of the one or more biomarkers as listed in step a) in said patient's blood serum or blood plasma sample to the concentration, expression level or activity of the one or more biomarkers as listed in step a) at the beginning of the therapy with the PDE4-inhibitor of formula A′,
   whereby steps a) and b) may be performed only once or may be repeated several times.

In a preferred embodiment, the one or more biomarker as mentioned in step a) is selected from the group consisting of KL-6, SP-D, MMP-7, CA-125, CA19-9, OPN, sICAM-1 and E-selectin.

In a preferred embodiment, the one or more biomarker as mentioned in step a) is selected from the group consisting of KL-6, SP-D, MMP-7 and E-selectin.

In a further preferred embodiment, the one or more biomarker as mentioned in step a) is selected from the group consisting of KL-6, SP-D and MMP-7.

In a particularly preferred embodiment, the one or more biomarker as mentioned in step a) is selected from the group consisting of KL-6 and SP-D.

In a particularly preferred embodiment, the one or more biomarker as mentioned in step a) is KL-6.

In another particularly preferred embodiment, the one or more biomarker as mentioned in step a) is SP-D.

In a further preferred embodiment the method of monitoring the treatment success is used in an PF-ILD-patient, preferably in an IPF-patient, who had been treated with 18 mg PDE4-inhibitor of formula A′ twice daily.

### 3. PHASE 2 TRIAL (NCT04419506)

### 3.1 Detailed Description of the Clinical Phase 2 Trial

Patients were aged ≥40 years and had a diagnosis of idiopathic pulmonary fibrosis based on the 2018 American Thoracic Society (ATS)/European Respiratory Society (ERS)/Japanese Respiratory Society/Latin American Thoracic Association guidelines (Raghu et al, Am J Respir Crit Care Med, 2018; 198: e44-e68). Patients with a usual interstitial pneumonia (UIP) or probable UIP pattern on high-resolution computed tomography consistent with idiopathic pulmonary fibrosis, confirmed by central review, were eligible (Raghu et al, AM j Respir Crit Care Med 2019; 200:1089-1092). Patients also had forced FVC ≥45% predicted, and diffusing capacity of the lungs for carbon monoxide (DLco) corrected for hemoglobin 25-<80% predicted. Patients were permitted to continue antifibrotic therapy (nintedanib or pirfenidone) if they had been receiving a stable dose for at least 8 weeks prior to screening. Patients with airways obstruction, recent respiratory tract infection, or a history of suicidal behavior in the past 2 years, were excluded.

The study was conducted in accordance with the principles of the Declaration of Helsinki and the Harmonized Tripartite Guidelines for Good Clinical Practice from the International Council for Harmonization and was approved by local authorities. The clinical protocol was approved by an independent ethics committee or institutional review board at each participating center. All patients provided written informed consent before study entry.

### 3.1.1 Study Design

The study was a double-blind, placebo-controlled, parallel-design, Phase 2 study performed at 90 sites in 22 countries. Patients were randomized 2:1 to receive either 18 mg of the PDE4 inhibitor of formula A′ twice daily or matching placebo for 12 weeks. After completion of the 12-week treatment period, patients entered a 1-week follow-up period. Patients who prematurely discontinued study medication were asked to attend all visits as originally planned to minimize missing data. Randomization was stratified according to use of background antifibrotics (no or yes) at baseline, targeting at least 60 patients per treatment group and up to 150 patients overall. Patients, investigators, central reviewers, and those involved in the study conduct and analysis were unaware of the treatment assignments. An interactive voice-response system was used to perform randomization.

The primary endpoint was change from baseline in FVC (mL) at 12 weeks. Spirometric results, assessed using spirometers provided by the sponsor (ERT SpiroSphere), were centrally reviewed to meet ATS/ERS criteria (Miller et al, Eur Respir J, 2005; 26: 319-338). The secondary endpoint was the percentage of patients with treatment-emergent adverse events. Change from baseline in Diffusion capacity or Transfer factor of the lung for carbon monoxide (DLco %) predicted corrected for hemoglobin was assessed as a further lung function efficacy endpoint using the site's own equipment and carried out according to ATS/ERS guidelines (Macintyre et al, Eur Respir J, 2005; 26: 720-735).

### 3.1.2 Statistical Analysis

The primary endpoint—change from baseline in FVC at Week 12—was evaluated separately in patients without and with background antifibrotics at baseline, and included all data collected while on treatment. The primary analysis was based on a Bayesian borrowing approach in order to incorporate historical data for the placebo arms within the groups treated without or with background antifibrotics via meta-analytic predictive priors that were made robust against prior-data conflicts (Schmidli et al, Biometrics 2014 70: 1023-1032). A vaguely informative prior was used for the arms using the PDE4 inhibitor of formula A′ (Compound of formula A′ in the following). The primary endpoint analysis was conducted in a two-step procedure. In the first step, the data from the current trial were analyzed with a restricted maximum likelihood-based approach using a mixed model with repeated measurements (MMRM). The analysis included the fixed, categorical effect of treatment at each visit, and the fixed, continuous effects of baseline FVC at each visit. Visit was treated as the repeated measure, with an unstructured covariance structure used to model the within-patient measurements. Based on this model, the adjusted mean changes from baseline in FVC at 12 weeks (and the related standard error) were calculated for the Compound of formula A′ and placebo arms within the patient groups without and with background antifibrotics. In the second step, the adjusted means in the placebo arms were combined with the meta-analytic predictive priors derived based on the clinical trials in the nintedanib clinical development program in idiopathic pulmonary fibrosis. In order to evaluate the treatment effects in each group, the posterior distribution for the treatment difference of the compound of formula A′ versus placebo with respect to the primary endpoint was used. The median of the posterior distribution for the treatment difference (and 95% credible intervals) was calculated as the primary analysis, and posterior probabilities that the treatment difference was higher than different boundaries were reported.

All treated patients were included in the safety analysis, stratified by use of background antifibrotics (no or yes), and the analysis was descriptive in nature. Safety was assessed by clinical and laboratory evaluation and the recording of treatment-emergent adverse events, as coded with the use of the Medical Dictionary for Regulatory Activities, version 22.0.

Missing data for the primary analysis (continuous endpoint) were not imputed. The MMRM analysis allows for missing data, assuming they are missing at random. Sensitivity analyses were conducted to investigate the potential effect of missing data as well as early discontinuation via a treatment policy strategy, and a pooled analysis combining all patients irrespective of background treatment with antifibrotics.

As this was an exploratory trial, no confirmatory testing and adjustment for multiplicity was planned. The sample size was chosen based on the evaluations of posterior probabilities for the change from baseline in FVC at Week 12, assuming a standard deviation of 200 mL and treatment differences of 70 mL and 20 mL in the patients treated without or with background antifibrotics, respectively.

Descriptive statistics were planned for the change from baseline in DLco at Week 12. An analysis based on the same MMRM as defined for the primary endpoint was conducted post hoc.

### 3.2 Results of the Clinical Phase 2 Trial

A total of 147 patients with idiopathic pulmonary fibrosis were randomized and treated with the compound of formula A′ or placebo. Demographic characteristics were comparable between the groups, although patients treated with background antifibrotics tended to have a longer time since diagnosis and lower FVC % predicted values at baseline.

A total of 15 patients discontinued the study. These patients were all in the arm obtaining the compound of formula A′ (5 and 10 patients without and with background antifibrotics, respectively). The primary reason for discontinuation was because of adverse events (3 and 10 patients without and with background antifibrotics, respectively). In patients without background antifibrotics, mean treatment durations were 81.4±12.3 and 85.6±3.8 days for the compound of formula A′ and placebo arms. For patients with background antifibrotics, this was 74.6±23.0 and 84.7±1.5 days, respectively.

### 3.2.1 Efficacy

The primary efficacy endpoint—the change from baseline in FVC at Week 12 based on a Bayesian borrowing approach using historical data—revealed that treatment with the compound of formula A′ stabilized lung function, in contrast to placebo, where there was a decline in FVC (FIG. 4).

In patients without background antifibrotics, the median change in FVC was +5.7 mL in the compound of formula A′ arm and −81.7 mL in the placebo arm (median difference: 88.4 mL, 99.8% probability that the compound of formula A′ is superior to placebo based on Bayesian borrowing analysis, see FIG. 4). In patients with background antifibrotics, these respective changes were +2.7 mL and −59.2 mL (median difference: 62.4 mL, 98.6% probability that the compound of formula A′ is superior to placebo based on Bayesian borrowing analysis) (FIG. 4).

The beneficial effects of the compound of formula A′ on FVC were also shown in the prespecified MMRM analysis, based on observed values only (FIG. 4, FIGS. 1, 2 and 3). The treatment effect estimated from the MMRM analysis was similar between the groups of patients without and with background antifibrotics, with an overall treatment effect of 88.4 mL (95% confidence interval [CI] 40.7 to 136.0) (FIG. 5). In the MMRM analysis, the mean change in FVC from baseline to Week 12 for the compound of formula A′ arms and placebo arms, respectively, was +6.1 mL and −95.6 mL for patients without background antifibrotics, corresponding to a difference of 101.7 mL (95% CI 25.0 to 178.4) (see FIG. 4 and FIG. 1), and +2.7 mL and −77.7 mL for patients with background antifibrotics, corresponding to a difference of 80.4 mL (95% CI 20.9 to 140.0) (see FIG. 4 and FIG. 2).

### 3.2.2 Safety

An overview of all Treatment-emergent adverse events is shown in Table 1. Table 2 shows the most frequently reported on-treatment adverse events reported in ≥3% of patients overall in the Compound of formula A′ treatment arms.

The proportion of patients with any adverse event was higher in the Compound of formula A′ treatment arm compared with the placebo arm for those without and with background antifibrotics. Adverse events leading to discontinuation were only reported in the Compound of formula A′ treatment arm.

The most common adverse events by organ class were gastrointestinal disorders, reported for 27.1% and 16.0% of patients without background antifibrotics, and 36.7% and 32.0% of patients with background antifibrotics in the Compound of formula A′ treatment arm and in the placebo arm, respectively (see Table 2). The most common adverse event by preferred term was diarrhea, which was also the most frequent adverse event leading to discontinuation. The proportion of patients with diarrhea was higher in the Compound of formula A′ treatment arm versus the placebo arm in the groups irrespective of background use of antifibrotics. Most cases of diarrhea were of mild intensity.
Severe adverse events were reported for 4.2% and 4.0% of patients without background antifibrotics, and for 4.1% and 4.0% of patients with background antifibrotics in the Compound of formula A′ treatment arm and in the placebo arm, respectively (see Table 1). Serious adverse events were reported for 6.3% and 20% of patients without antifibrotics and 6.1% and 0% of patients with background antifibrotics, respectively (see Table 1). There were 2 patients with fatal adverse events in the Compound of formula A′ treatment arm: COVID pneumonia (without background antifibrotics) and one case of suspected vasculitis and suspected IPF exacerbation in a patient with background antifibrotics, of which vasculitis was not confirmed by an independent data monitoring committee (with background antifibrotics).

TABLE 1
Treatment-emergent adverse events

| | Without background | | With background | |
|---|---|---|---|---|
| | antifibrotics | | antifibrotics | |
| | Compound of | | Compound of | |
| | formula A' | Placebo | formula A' | Placebo |
| Adverse event | (N = 48) | (N = 25) | (N = 49) | (N = 25) |
| Any adverse event | 31 (64.6) | 13 (52.0) | 36 (73.5) | 17 (68.0) |

Most frequent adverse event (>10% in at least one arm)

| | | | | |
|---|---|---|---|---|
| Diarrhea | 8 (16.7) | 2 (8.0) | 15 (30.6) | 4 (16.0) |
| Fatigue | 2 (4.2) | 1 (4.0) | 1 (2.0) | 3 (12.0) |
| Severe adverse events | 2 (4.2) | 1 (4.0) | 2 (4.1) | 1 (4.0) |
| Investigator-defined | 9 (18.8) | 5 (20.0) | 18 (36.7) | 5 (20.0) |
| drug-related | | | | |
| adverse events | | | | |
| Adverse events leading | 3 (6.3) | 0 (0.0) | 10 (20.4) | 0 (0.0) |
| to discontinuation | | | | |
| of study drug | | | | |

Most frequent adverse event leading to discontinuation (>5% of patients)

| | | | | |
|---|---|---|---|---|
| Diarrhea | 0 (0.0) | 0 (0.0) | 3 (6.1) | 0 (0.0) |
| Patients with | 0 (0.0) | 0 (0.0) | 1 (2.0) | 0 (0.0) |
| pre-specified | | | | |
| adverse events | | | | |
| of special interest* | | | | |

Serious adverse events^{†}

| | | | | |
|---|---|---|---|---|
| All | 3 (6.3) | 5 (20.0) | 3 (6.1) | 0 (0.0) |
| Resulted in death | 1 (2.1) | 0 (0.0) | 1 (2.0) | 0 (0.0) |
| Required or prolonged | 2 (4.2) | 3 (12.0) | 3 (6.1) | 0 (0.0) |
| hospitalization | | | | |
| Other comparable | 1 (2.1) | 2 (8.0) | 0 (0.0) | 0 (0.0) |
| medical criteria | | | | |

*Adverse events of special interest were vasculitis and hepatic injury.
^{†}There were no reported cases for: immediate life threatening, persistent or significant disability/incapacity or congenital anomaly/birth defect. All results are shown as n (%).

TABLE 2
Most frequently reported on-treatment adverse events*

| | Without background | | With background | |
|---|---|---|---|---|
| | antifibrotics | | antifibrotics | |
| | Compound of | | Compound of | |
| System organ | formula A' | Placebo | formula A' | Placebo |
| class/preferred term | (N = 48) | (N = 25) | (N = 49) | (N = 25) |
| Gastrointestinal disorders | 13 (27.1) | 4 (16.0) | 18 (36.7) | 8 (32.0) |
| Diarrhea | 8 (16.7) | 2 (8.0) | 15 (30.6) | 4 (16.0) |
| Flatulence | 3 (6.3) | 1 (4.0) | 2 (4.1) | 1 (4.0) |
| Dyspepsia | 3 (6.3) | 0 (0.0) | 2 (4.1) | 1 (4.0) |
| Nausea | 2 (4.2) | 2 (8.0) | 1 (2.0) | 0 (0.0) |
| Vomiting | 1 (2.1) | 1 (4.0) | 2 (4.1) | 1 (4.0) |
| Respiratory thoracic and | 8 (16.7) | 2 (8.0) | 6 (12.2) | 4 (16.0) |
| mediastinal disorders | | | | |
| Cough | 3 (6.3) | 1 (4.0) | 4 (8.2) | 2 (8.0) |
| Dyspnea | 3 (6.3) | 0 (0.0) | 0 (0.0) | 0 (0.0) |
| Nervous system disorder | 4 (8.3) | 2 (8.0) | 9 (18.4) | 3 (12.0) |
| Headache | 3 (6.3) | 0 (0.0) | 3 (6.1) | 1 (4.0) |
| Infections and | 6 (12.5) | 3 (12.0) | 7 (14.3) | 2 (8.0) |
| infestations | | | | |
| Nasopharyngitis | 0 (0.0) | 0 (0.0) | 4 (8.2) | 0 (0.0) |
| General disorders and | 9 (18.8) | 4 (16.0) | 7 (14.3) | 4 (16.0) |
| administration site | | | | |
| conditions | | | | |
| Asthenia | 3 (6.3) | 0 (0.0) | 1 (2.0) | 0 (0.0) |
| Fatigue | 2 (4.2) | 1 (4.0) | 1 (2.0) | 3 (12.0) |
| Metabolism and nutrition | 1 (2.1) | 2 (8.0) | 3 (6.1) | 0 (0.0) |
| disorders | | | | |
| Decreased appetite | 1 (2.1) | 0 (0.0) | 2 (4.1) | 0 (0.0) |
| Investigations | 5 (10.4) | 2 (8.0) | 10 (20.4) | 5 (20.0) |
| Weight decreased^{†} | 2 (4.2) | 0 (0.0) | 1 (2.0) | 0 (0.0) |

*Reported in ≥ 3% of patients overall in the Compound of formula A' arms;
^{†}The mean observed weight loss in the group without antifibrotics in patients treated with the Compound of formula A' and placebo, respectively, was −0.76 and −0.31, and in the group with antifibrotics −1.41 kg and −1.07, over 12 weeks

### 3.2.3 Biomarker Analysis During the Clinical Phase 2 Trial

Blood samples were collected from patients during the clinical phase 2 trial at baseline, at 2, 4, 8 and 12 weeks (after baseline) of the “Compound of formula A′ treatment arm” and of the “Placebo arm”, and blood plasma and/or blood serum was prepared according to methods known in the art.

Fibrosis-related biomarkers were assessed in plasma or serum using ELISA or Luminex technologies. The changes in these fibrosis-related biomarkers at weeks 2, 4, 8 and 12 after baseline were analysed using mixed models with repeated measures (MMRM). The fold changes in the tested biomarkers over baseline over time were then compared between patients of the of the “Compound of formula A′ treatment arm” and patients of the “Placebo arm”.

Biomarker values were only considered valid up to +2 days for CRP and up to +7 days after last drug intake for all other proteins.

Some of the tested IPF-related protein biomarkers mainly seem to be related with the epithelial/endothelial barrier integrity such as:
Krebs von den Lungen protein (KL-6): a glycoprotein expressed on the extracellular surface of type II alveolar epthelial cells (AECs) and in bronchiolar epithelial cells in the lung largely studied in ILDs due to its overexpression in affected lung and regenerating type II AECs. KL-6 is increased in serum of several ILDs including IPF. Zhang et al, Front. Immunol. (2021), 12: 745233 discusses KL-6 elevated levels/threshold levels in serum that could predict the progression/mortality of ILD/IPF, such as a serum KL-6 level of >1273 U/ml as the most reliable predictor of end-stage lung disease development or a serum KL-6 level of >933 U/ml showing a reduced survival than those without such a high KL-6 level.
Pulmonary surfactant protein D (SP-D) is expressed in alveolar type II and bronchiolar epithelial cells and is secreted into alveoli and conducting airways. However, SP-D has also been measured in serum and is increased in patients with acute respiratory distress syndrome, pulmonary fibrosis, and alveolar proteinosis (Pan et al, Am J Physiol Lung Cell Mol Physiol 2002; 282 (4): L824-32).
CA-125 (also named MUC-16) is a high-molecular weight glyco-protein that is expressed by the various epithelial cell surfaces of the human body (Haridas et al, The FASEB J, Vol. 28, pp. 4184-99). It is still employed as an effective marker for early epithelial ovarian cancer dectection.
Carbohydrate antigen 19-9 (CA19-9) is a type of glycoproteins located in epithelium of pancreatic and bile ducts (Wang et al, Oncotarget, 2017, 8: 2164-2170).

Some of the tested IPF-related protein biomarkers mainly seem to be related with the extracellular matrix (ECM) turnover:
Matrilysin—also known as matrix metalloproteinase 7 (MMP7) is known to degrade several ECM components in normal physiological processes, such as embryonic development, reproduction, tissue remodeling, as well as disease processes such as arthritis and metastasis. In baseline BUILD-3 samples (BUILD=Bosentan use in Interstitial Lung Disease), only MMP-7 showed clearly elevated protein levels in IPF patients compared with samples from healthy controls and further investigations demonstrated that MMP-7 levels also increased over time. (Bauer et al ERJ Open Res 2017; 3: 00074-2016). The geometric mean serum MMP-7 concentrations and p-values in comparison with the healthy control group (1.25 ng/mL) were: IPF at baseline: 2.25 ng/mL (p<0.0001), IPF (4 month): 1.97 ng/mL (p<0.01) and IPF at end of study: 2.64 ng/mL (p<0.0001) (Bauer et al ERJ Open Res 2017; 3: 00074-2016).
Stromelysin—also known as matrix metalloproteinase 3 (MMP3) is known to degrade several ECM components
Cartilage oligomeric matrix protein (COMP)—also known as thrombospondin-5, is an extracellular matrix (ECM) protein primarily present in cartilage. In humans it is encoded by the COMP gene (Udomsinprasert et al, Sci Rep (2021) 11: 16695).
Prostasin—also known as channel activating protease 1) is an extracellular serine protease with trypsin-like activity which cleaves synthetic substrates in vitro, preferentially at carboxy-terminal side of arginine residue (Aggarwal et al, Biomark. 2013, 2013:179864).
Von-Willebrand-Factor (vWF) is a useful biomarker for liver fibrosis and prediction of hepatocellular carcinoma development in patients with hepatitis B and C (Takaya et al, United European Gastroenterol. 2018; 6 (9): 1401-1409). However, Von-Willebrand-Factor has not been known to be a biomarker for PF-ILD or IPF.

Some of the tested IPF-related protein biomarkers seem to play mainly a role in inflammation:
C-reactive protein (CRP) has been reported to be a biomarker for the prediction of the severity of pulmonary exacerbations in patients with cystic fibrosis (Giron-Moreno et al, BMC Pulm Med. 2014, 14: 150).
Soluble Intercellular adhesion molecule 1 (sICAM1) have been reported to increase in patients with idiopathic pulmonary fibrosis (Okuda et al, Springerplus 2015; 4: 657)

### 3.2.3.1 Methods

### Description of ELISA (MLM)

At MLM (MLM Medical Labs GmbH, Mönchengladbach, Germany, www.mlm-labs.com) the biomarker KL-6 (Krebs von den Lungen) was measured using CLEIA Test kits (Lumipulse® G KL-6 Immunoreaction Cartridges) from Fujirebio (www.fujirebio.com) and a Lumipulse G1200 system, the biomarker sICAM-1 was measured using an ECLIA assay (V-PLEX Plus Vascular Injury Panel 2 Human Kit) from Meso Scale Discovery (MSD) on the Meso QuickPlex SQ120 device, and the biomarker MMP-7 was measured using a Quantikine® ELISA assay (Human MMP-7/PARC Quantikine® ELISA kit) from R&D Systems on a TECAN Absorbance Reader, according to manufacturer's instructions.

### Description of Luminex Technology MyriadRBM)

The biomarkers CA-125, CA19-9, COMP, CRP, E-selectin, sICAM-1, MMP-3, MMP-7, Prostasin, TIMP-1, and vWF were measured using validated microsphere-based immune-multiplexing assays using Luminex technology at MyriadRBM (Rules Based Medicine, a q2 solutions company, Austin, Texas, USA, www.rbm.q2labsolutions.com).

### 3.2.3.2 Results

The biomarker data as collected from the different treatment groups of the phase II trial have been analysed with regard to three different aspects:
1. The biomarker data has been analysed with regard to the “pharmacodynamic potential” of each respective biomarker, that means the change from baseline (CfB) of the biomarker level over time during treatment with the compound of formula A′ has been analysed. Biomarkers which prove to have a “pharmacodynamic potential” during treatment with the compound of formula A′ therefore could be used to determine and quantify the molecular and physiological effect of the compound of formula A′ during treatment.
2. The biomarker data has been analysed with regard to the “prognostic potential” of each respective biomarker, that means the level of biomarker at baseline vs. clinical outcome after 12 weeks (=the absolute FVC [mL] change from baseline at week 12 and the absolute change from baseline in Dlco % predicted at week 12) in the Placebo groups has been analysed which is disease-related and may be used to assess the progression of the disease (e.g. fast vs. slow progressors).
3. The biomarker data has been analysed with regard to their “outcome-related potential” of each respective biomarker, that means the change from baseline (CfB) in biomarker level vs. the clinical outcomes after 12 weeks (=the absolute FVC [mL] change from baseline at week 12 and the absolute change from baseline in Dlco % predicted at week 12) in the compound of formula A′ treatment groups has been analysed. Hereby, a potential early prediction of both clinical endpoint and treatment effect may be achieved.

Table 3 summarizes the adjusted mean (95% Confidence interval) for fold change from baseline in various protein biomarkers (based on the Mixed model repeated measurement model) in the blood of patients without antifibrotic background treatment (non-AF-background) after 4 and after 12 weeks of treatment with the compound of formula A′.

A lower fold change from baseline over time (that means “decrease over time”) for the “non-AF-background-patients” from the “Compound of formula A′ treatment arm” compared to the patients from the “Placebo arm” could be measured for the following protein biomarkers: KL-6, SP-D, CA-125 (MUC-16), Matrilysin (=MMP7), COMP, Prostasin, E-selectin and vWF (see Table 3 and FIG. 6, 8, 10, 14, 18, 20, 22, 24, 28). A higher fold change from baseline over time (that means “increase over time”) for the “non-AF-background-patients” from the “Compound of formula A′ treatment arm” compared to the patients from the “Placebo arm” could be measured for the protein biomarker CRP (see Table 3 and FIG. 26) after 4 and after 12 weeks of treatment with the compound of formula A′.

Table 4 summarizes the adjusted mean (95% Confidence interval) for fold change from baseline in various protein biomarkers (based on the Mixed model repeated measurement model) in the blood of patients with antifibrotic background treatment (AF-background).

A lower fold change from baseline over time (that means “decrease over time”) for the “AF-background-patients” from the “Compound of formula A′ treatment arm” compared to the patients from the “Placebo arm” could be measured for the following protein biomarkers: KL-6, SP-D, Matrilysin (=MMP7), Prostasin and E-selectin (see Table 4 and FIG. 7, 9, 15, 21, 29). A higher fold change from baseline over time (that means “increase over time”) for the “AF-background-patients” from the “Compound of formula A′ treatment arm” compared to the patients from the “Placebo arm” could be measured for the protein biomarker CRP (see Table 4 and FIG. 27).

TABLE 3
Fold changes from baseline to Week 4 and to Week 12 of various biomarkers in the
blood of patients without antifibrotic background treatment (Non-AF-background)

| | Compound of formula A′ | | Placebo | | Compound of formula A′ | |
|---|---|---|---|---|---|---|
| | Fold change from baseline | | Fold change from baseline | | Fold change from | |
| | to Week 4*[1] | | to Week 4*[1] | | baseline to Week 12*[1] | |
| | Adjusted | | Adjusted | | Adjusted | |
| | mean (95% | | mean (95% | | mean (95% | |
| | confidence | Adjusted | confidence | Adjusted | confidence | Adjusted |
| Biomarker | interval) | p-value | interval) | p-value | interval) | p-value |
| KL-6 (U/mL) | 0.911 | <0.0001 | 0.997 | 0.9092 | 0.916 | 0.0017 |
| SP-D (μg/L) | 0.833 | <0.0001 | 1.024 | 0.5677 | 0.875 | 0.0004 |
| CA-125 (MUC-16) | 0.956 | 0.5943 | 1.143 | 0.1909 | 1.039 | 0.6863 |
| (U/mL) | | | | | | |
| CA19-9 (U/mL) | 0.901 | 0.1651 | 1.014 | 0.8749 | 1.016 | 0.8433 |
| Matrilysin (μg/L) | 0.908 | 0.0079 | 0.971 | 0.5091 | 0.883 | 0.0122 |
| (MMP7, MLM) | | | | | | |
| Stromelysin-1 | 0.963 | 0.3052 | 1.031 | 0.4890 | 0.960 | 0.3542 |
| MMP3) (μg/L) | | | | | | |
| COMP (μg/L) | 0.970 | 0.4044 | 1.042 | 0.3556 | 0.949 | 0.1836 |
| Prostasin (μg/L) | 0.883 | <0.0001 | 1.006 | 0.8177 | 0.905 | 0.0009 |
| vWF (μg/L) (mg/L) | 0.991 | 0.8917 | 1.197 | 0.0199 | 0.972 | 0.6299 |
| sICAM1 (RBM) | 0.956 | 0.3658 | 0.937 | 0.3085 | 0.937 | 0.1691 |
| (μg/L) | | | | | | |
| CRP (mg/L) | 2.134 | <0.0001 | 1.101 | 0.6640 | 1.479 | 0.0257 |
| E-selectin (μg/L) | 0.943 | 0.0882 | 1.041 | 0.3294 | 0.982 | 0.5316 |

| | Placebo | | | |
|---|---|---|---|---|
| | Fold change from | | | |
| | baseline to Week 12*[1] | | | |
| | Adjusted | | | |
| | mean (95% | | See | Change of Biomarker from |
| | confidence | Adjusted | FIG. | baseline to Week 12 in Comp. |
| Biomarker | interval) | p-value | No. | A′ arm vs. Placebo arm |
| KL-6 (U/mL) | 0.962 | 0.2504 | 6 | ↓ (significant decrease) |
| SP-D (μg/L) | 0.972 | 0.5306 | 8 | ↓ (significant decrease) |
| CA-125 (MUC-16) | 1.186 | 0.1409 | 10 | ↓ (minor decrease) |
| (U/mL) | | | | |
| CA19-9 (U/mL) | 1.010 | 0.9212 | 12 | ↓ |
| Matrilysin (μg/L) | 1.008 | 0.8983 | 14 | ↓ (significant decrease) |
| (MMP7, MLM) | | | | |
| Stromelysin-1 | 0.957 | 0.4217 | 16 | ↓ |
| MMP3) (μg/L) | | | | |
| COMP (μg/L) | 1.070 | 0.1610 | 18 | ↓ |
| Prostasin (μg/L) | 0.995 | 0.8811 | 20 | ↓ |
| vWF (μg/L) (mg/L) | 1.128 | 0.0873 | 22 | ↓ |
| sICAM1 (RBM) | 0.954 | 0.4406 | 24 | — |
| (μg/L) | | | | |
| CRP (mg/L) | 1.589 | 0.0292 | 26 | ↑ (significant increase) |
| E-selectin (μg/L) | 0.961 | 0.2670 | 28 | ↓ |

*back transformation to the original scale. Adjusted mean ratio in fold change is presented for treatment comparison.
[1]based on MMRM model, with fixed categorical effects of treatment at each visit and the fixed continuous effects of baseline value of the protein biomarker at each visit and age,

TABLE 4
Fold changes from baseline to Week 4 and to Week 12 of various biomarkers in
the blood of patients with antifibrotic background treatment (AF-background)

| | Compound of formula A′ | | Placebo | | Compound of formula A′ | |
|---|---|---|---|---|---|---|
| | Fold change from baseline | | Fold change from | | Fold change from baseline | |
| | to Week 4*[1] | | baseline to Week 4*[1] | | to Week 12*[1] | |
| | Adjusted | | Adjusted | | Adjusted | |
| | mean (95% | | mean (95% | | mean (95% | |
| | confidence | Adjusted | confidence | Adjusted | confidence | Adjusted |
| Biomarker | interval) | p-value | interval) | p-value | interval) | p-value |
| KL-6 (U/mL) | 0.960 | 0.0035 | 0.979 | 0.2590 | 0.923 | 0.0030 |
| SP-D (μg/L) | 0.879 | <0.0001 | 0.949 | 0.1399 | 0.894 | 0.0078 |
| CA-125 (MUC-16) | 1.030 | 0.6736 | 1.105 | 0.2728 | 1.067 | 0.4566 |
| (U/mL) | | | | | | |
| CA19.9 (U/mL) | 0.987 | 0.8477 | 0.933 | 0.4459 | 0.914 | 0.0718 |
| Matrilysin (μg/L) | 0.941 | 0.0665 | 0.946 | 0.2110 | 0.981 | 0.6244 |
| (MMP7, MLM) | | | | | | |
| Stromelysin-1 | 1.063 | 0.0450 | 1.008 | 0.8460 | 1.056 | 0.0462 |
| MMP3) (μg/L) | | | | | | |
| COMP (μg/L) | 0.991 | 0.7659 | 0.981 | 0.6161 | 1.023 | 0.4349 |
| Prostasin (μg/L) | 0.939 | 0.0125 | 0.992 | 0.7921 | 0.933 | 0.0086 |
| vWF (mg/L) | 1.035 | 0.4628 | 0.996 | 0.9448 | 0.964 | 0.4379 |
| sICAM1 (RBM) | 1.049 | 0.3516 | 1.084 | 0.2250 | 1.069 | 0.3415 |
| (μg/L) | | | | | | |
| CRP (mg/L) | 1.780 | <0.0001 | 1.291 | 0.1234 | 1.545 | 0.0021 |
| E-selectin (μg/L) | 0.933 | 0.0053 | 1.020 | 0.5258 | 0.936 | 0.0604 |

| | Placebo | | | |
|---|---|---|---|---|
| | Fold change from | | | |
| | baseline to Week 12*[1] | | | |
| | Adjusted | | | |
| | mean (95% | | See | Change of Biomarker from |
| | confidence | Adjusted | FIG. | baseline to Week 12 in Comp. |
| Biomarker | interval) | p-value | No. | A′ arm vs. Placebo arm |
| KL-6 (U/mL) | 1.002 | 0.9515 | 7 | ↓ (significant decrease) |
| SP-D (μg/L) | 1.074 | 0.1667 | 9 | ↓ (significant decrease) |
| CA-125 (MUC-16) | 0.935 | 0.5314 | 11 | |
| (U/mL) | | | | |
| CA19.9 (U/mL) | 0.938 | 0.2922 | 13 | |
| Matrilysin (μg/L) | 0.952 | 0.3580 | 15 | ↓ |
| (MMP7, MLM) | | | | |
| Stromelysin-1 | 1.043 | 0.2184 | 17 | |
| MMP3) (μg/L) | | | | |
| COMP (μg/L) | 0.963 | 0.3003 | 19 | |
| Prostasin (μg/L) | 1.007 | 0.8296 | 21 | ↓ |
| vWF (mg/L) | 1.016 | 0.7932 | 23 | |
| sICAM1 (RBM) | 1.077 | 0.4014 | 25 | — |
| (μg/L) | | | | |
| CRP (mg/L) | 1.303 | 0.1231 | 27 | ↑ (significant increase) |
| E-selectin (μg/L) | 1.066 | 0.1460 | 29 | ↓ |

*back transformation to the original scale. Adjusted mean ratio in fold change is presented for treatment comparison.
[1]based on MMRM model, with fixed categorical effects of treatment at each visit and the fixed continuous effects of baseline value of the protein biomarker at each visit and age,

3.2.4 Analysis of “Prognostic Potential” of Different Biomarkers for Disease Progression Based on FVC Decline from the INBUILD Study

As an example, the prognostic potential for disease progression of various biomarkers was analyzed in the Placebo arm of the INBUILD study, a Phase III trial testing Nintedanib in PF-ILD patients (Wells et al., LancetRespiMed 2020, Nintedanib in patients with progressive fibrosing interstitial lung diseases—subgroup analyses by interstitial lung disease diagnosis in the INBUILD trial: a randomised, double-blind, placebo-controlled, parallel-group trial, NCT02999178). Several biomarkers in peripheral blood, including KL-6, MMP7, sICAM-1, CA19-9, were shown to be prognostic for disease progression using annual rate of decline in FVC over 52 weeks as a measure of disease progression.

As an example, the cut-off values for some of the prognostic biomarkers are shown in Table 5, and the annual rate of decline in FVC over 52 weeks (mean and 95% Confidence Intervals) is given for the respective subgroups showing a significant difference in rate of FVC decline (as a measure of disease progression) for these subgroups. The cut-off values are to be seen in the context of the INBUILD study and its patients cohort and are shown as examples.

TABLE 5
“Prognostic Potential” of biomarkers KL-6, CA-19-9, MMP7 and
sICAM1 in PF-ILD patients of the INBUILD trial:

| Biomarker/ | | | Annual rate of decline in | |
|---|---|---|---|---|
| Population | Subgroups | N | FVC (mL/yr) over 52 weeks | p-value |
| KL-6 | ≤1120 U/mL | 144 | −184.01 (−227.23, −140.79) | |
| UIP-like | >1120 U/mL | 52 | −283.68 (−358.76, −108.61) | 0.0245 |
| CA 19-9 | ≤13.5 U/mL | 108 | 150.09 (−195.61, −104.56) | 0.0249 |
| Overall | >13.5 U/mL | 179 | −216.50 (−251.64, −181.36) | |
| MMP7 | ≤7.02 μgEq/L | 77 | −139.73 (−193.55, −85.92) | |
| Overall | >7.02 μgEq/L | 235 | −207.51 (−238.45, −176.58) | 0.0335 |
| sICAM1 | ≤662 ng/ml | 156 | −150.08 (−187.24, −112.93) | |
| Overall | >662 ng/ml | 156 | −232.71 (−271.00, −194.42) | 0.0025 |

As further examples, the prognostic potential for disease progression of KL-6, CA19-9, sICAM-1 and MMP7 (among others) was shown in a variety of cohorts of IPF and PF-ILD patients and cut off levels have been determined in some instances which have to be seen in context of the respective cohort. The listed references provide an overview:
Adegunsoye et al., Chest 2020, Circulating plasma biomarkers of survival in AF-treated patients with IPF
Alqalyoobi et al., AJRCCM 2020, Circulating plasma biomarkers of progressive Interstitial Lung Diseases
Bowman et al., FrontinMed 2021, Biomarkers in Progressive Fibrosing Interstitial Lung Disease: Optimizing diagnosis, prognosis and treatment response
Choi et al., RespiRes 2022, Blood KL-6 levels predict treatment response to antifibrotic therapy in patients with IPF
Clynick et al., ERJ 2021, Biomarker signatures for progressive IPF
Daccord and Maher, F1000Research 2016, Recent advances in understanding IPF
Guiot et al., Lung 2017, Blood biomarkers in IPF
Maher et al., LancetRespiMed 2017, An epithelial biomarker signature for IPF (PROFILE)
Jee et al., PharmacolTherap 2019, Review: serum biomarkers in IPF and SSc-ILD—frontiers and horizons
Jenkins et al., LancetRespiMed 2015, Longitudinal change in collagen degradation biomarkers in IPF (PROFILE)
Neighbors et al., LancetRespiRes 2018, Prognostic and predictive biomarkers for patients with IPF (CAPACITY and ASCEND)
Richards et al., AJRCCM 2012, Peripheral blood proteins predict mortality in IPF
Rosas et al., PLoS Med 2008, MMP1 and MMP7 as potential peripheral blood biomarkers in IPF.

### 3.2.5 Summary of Biomarker Analyses

### 3.2.5.1 “Pharmacodynamic Potential”

In order to analyse the “pharmacodynamic potential” of each tested biomarker for the treatment with compound of formula A′ the change from baseline (CfB) of the respective biomarker over time during treatment with the compound of formula A′ has been determined in the “active agent arms” (“treatment arms”) of the Compound of formula A′ phase II trial (48 patients in the “active arm” of the “AF-group” and 43 patients in the “active arm” of the “non-AF-group”).

An MMRM analysis on the biomarker change overtime during treatment with the compound of formula A′ has been performed. The result of this MMRM analysis on the change overtime for different biomarkers during treatment with the compound of formula A′ is shown in FIG. 30. FIG. 30 shows a descriptive boxplot for biomarker changes from baseline (CfB) overtime during “Compound of formula A′ treatment”. Decreases in the respective biomarkers overtime during treatment with the compound of formula A′ are depicted in violet/blue colour shades and increases in the respective biomarkers overtime during treatment with the compound of formula A′ are depicted in orange/red colour shades. No changes in biomarkers overtime are depicted in white.

The pharmacodynamic potentials of the different biomarkers as tested are mostly clear and consistent within the “non-AF-group”.

From FIG. 30 it is evident that a treatment with compound of formula A′ substantially decreases the levels of the biomarkers SP-D (=PSP-D), MMP7 (=Matrilysin, as shown in both, the RBM- and the MLM-assay) and KL-6, regardless whether background therapy is additionally administered or not. The biomarkers SP-D, MMP7 (=Matrilysin) and KL-6 therefore have a clear “pharmacodynamic potential” during treatment with the compound of formula A′. Thus, SP-D, MMP-7 and KL-6 could be used to determine and quantify the molecular and physiological effect of the compound of formula A′ during treatment.

Further, FIG. 30 shows that treatment with compound of formula A′ seems to be associated with an increase of the levels of C-reactive protein (CRP).

### 3.2.5.2 “Prognostic Potential”

In order to analyse the “prognostic potential” of each tested biomarker the changes from baseline (CfB) of the clinical endpoints (=Forced Vital Capacity (FVC) and DLCO % pred) at week 12 vs. Baseline biomarker levels from the Placebo-arms of the “AF-group” (25 patients) and of the “non-AF-group” (25 patients) of phase II trials were analysed and compared.

FIG. 31 contains a scatterplot that was generated for the CfB of the FVC-value vs. baseline biomarker value and for the CfB of the DLCO % pred-value vs. baseline biomarker value.

A rank-based correlation analysis was performed. FIG. 31 shows that prognostic signals are mostly clear in the non-AF-group and that the biomarkers SP-D, KL-6, CA-125 and sICAM-1 (MLM) all show a “prognostic potential” in IPF and therefore may be used to assess the progression of IPF (fast vs. slow progressors).

Table 5 which includes biomarker analysis data from patients of the INBUILD trial shows as an example additionally that biomarkers KL-6, CA 19-9, MMP7 and sICAM1 all show a “prognostic potential” for disease progression and therefore may be used to assess the progression of PF-ILD.

### 3.2.5.3 “Outcome-Related Potential”

In order to find out which biomarker is “outcome-related” the correlation between the change in biomarkers and the change in clinical endpoint (=CfB FVC and CfB DLCO) with a focus on both active arms (that means the “treatment-arms with the compound of formula A′ in the AF-group and in the non-AF-group) was analyzed. An “outcome-related biomarker” has the potential to be used for an early prediction of both, the clinical endpoint and a treatment effect with the active agent (here: the compound of formula A′).

FIG. 32 shows a scatterplot between the CfB biomarker values vs. the CfB FVC at week 12 and the CfB DLCO at week 12 for all compound of formula A′ treatments arms (AF-group, non-AF-group and pooled group). As shown in FIG. 32 the change in the biomarkers KL-6, SP-D, E-selectin and sICAM-1 shows a potential association with the CfB of the FVC after 12 weeks of treatment with the compound of formula A′.

### 3.2.5.4 Summary of the Biomarker Analyses

In chapter 1.1.2.1 it was described that the compound of formula A′ seems to decrease the levels of biomarkers KL-6, SP-D and MMP7. Further, an approximately 2× elevation of the CRP-levels could be observed in the compound of formula A′ treatment groups regardless of background therapy. Consequently, this hints at the existence of a “pharmacodynamic potential” of the biomarkers KL-6, SP-D and MMP7 (Matrilysin) during treatment with the compound of formula A′. KL-6, SP-D and MMP7 (Matrilysin) could therefore be used to determine and quantify the molecular and physiological effect of the compound of formula A′ during treatment (see FIG. 33).

In chapter 1.1.2.2 it was described that the biomarkers KL-6, SP-D, CA-125 and sICAM-1 seem to show a “prognostic potential” for IPF and could therefore be used to assess the progression of IPF (fast vs. slow progressors), (see FIG. 33).

In chapter 1.1.2.4 it was described that biomarkers KL-6, sICAM-1, SP-D and E-selectin seem to have an “outcome-related potential”, meaning that a high baseline level of any of the biomarkers KL-6, sICAM-1, SP-D and E-selectin shows a potential correlation to the decrease-from-baseline in lung function at week 12, measured as CfB in FVC after 12 weeks or as CfB in Dlco % pred. at week 12, during the treatment of an IPF patient with the compound of formula A′ (see FIG. 33).

FIG. 33 summarizes the findings in so far as only the biomarkers KL-6 and SP-D seem to combine all three properties: they are
“outcome-related biomarkers”
with a pharmacodynamic potential
and with a prognostic potential.

Additionally FIG. 33 shows that biomarker sICAM-1 does not have a pharmacodynamic potential but combines a prognostic potential for IPF and is an “outcome-related biomarker” with regard to treatment with the compound of formula A′.

### 4. IN VIVO STUDY CONCERNING THE EFFICACY OF THE PDE4-INHIBITOR OF FORMULA A′ IN A RAT BLEOMYCIN MODEL OF PULMONARY FIBROSIS

### 4.1 Summary

Pulmonary remodeling was induced in rats by a single intratracheal administration of 1 mg/kg bleomycin. Oral administration of 2.5 mg/kg of the phosphodiesterase inhibitor (PDE4) of formula A′ administered twice daily (0 and 12 hours) exhibited a 64% improvement in tissue volume as measured by micro computed tomography (μCT).

This was accompanied by an improvement in lung function parameters of airway resistance, compliance, and airspace volume (measured under 30 cm H₂O pressure), and by a reduction of total Osteopontin (OPN) protein levels in lung tissue.

### 4.2 Method

Adult, test-naïve, male Wistar rats (WI(Han)); 280-300 g) were purchased from Janvier (Janvier Labs, Le Genest-Saint-Isle, France). All animal experimentation was conducted in accordance with German national guidelines and legal regulations and approved by the ethical committee Regierungspräsidium Tübingen (Germany) (Permit Number: 12-012).

Bleomycin was dissolved in phosphate buffered saline (PBS) to a final concentration of 1.5 mg/mL. On day 0, animals were transiently anaesthetised with isoflurane (3-4%) in oxygen. Using a 1 ml-Syringe with a 22-gauge flexible cannula, 200 μL/kg of either saline or bleomycin (final dose 1 mg/kg) were administered intratracheally.

The PDE4-inhibitor of formula A′ was dissolved in 0.5% hydroxyethyl cellulose containing 0.01% Tween20 and applied by oral gavage at a dose of 2.5 mL/kg twice daily at 06:00 and 18:00 from day 10 until day 20.

On day 18, animals were anaesthetized using 3-4% isoflurane and placed on the μCT (Quantum FX) scanner mounting plate. After measurement, animals were returned to the home cage for recovery. During imaging, synchronization procedures were employed for avoidance of motion blur (respiration, heart beat) thereby improving the quality of the images. The reconstruction of the three-dimensional pictures from the raw data could subsequently be performed.

Animals were anaesthetised with Narcoren (60 mg/mL/kg body weight i.p.) and Ketamine (0.5-1 mg/animal, i.m.). The trachea was exposed, a small incision made, and a tracheal tube fixed within the trachea by a ligature. Spontaneous breathing was suppressed by pancuronium (0.8 mg/kg; 1 mL/kg body weight, i.v.) and animals were mechanically ventilated with a tidal volume of 10 mL/kg, breathing frequency of 90 breaths/minute and a positive end expiratory pressure of 3 cmH₂O. Airway resistance, compliance, elastance and pressure volume loop was determined by means of a FlexiVent system supported by software version 7.2. To perform the lung function measurement the template “FlexiVent FX-Rat Default-rel.B” with the script “Rat 6basic_1loop v7.0 Lamb” was used. A deep inspiration to total lung capacity (Deep Inflation) was performed with an inflation pressure limited to 30 cmH₂O. A “Snapshot-90 v7.0, Quick Prime-3 v7.0, PVS-V v7.0 and PVs-P v7.0 measurement was subsequently conducted. After the lung function measurement animals were euthanized by an overdose of Narcoren (0.16 g/5 ml, 1 ml/animal i.v.).

The right main bronchus was occluded with a ligature and the right lung was removed.

Right lung tissue was homogenized using an OmniPrep into PBS+1% BSA+0.714% Protease inhibitor (100 mg tissue per ml). The homogenate was centrifuged for 10 min at 4° C. at 4500 rpm and 3×200 μL of the supernatant plus the pellet was stored at −80° C.

OPN ELISA (R&D Systems, #MOST00) was performed according to the instruction manual.

All data are presented as means±S.E.M. of n animals. Statistical differences between groups were analyzed by the non-clinical statistical group.

### 4.3 Results

Airway mechanics could be assessed by pressure-volume loops, which is a measurement of airway volume as the pressure is incrementally increased and then decreased. Following bleomycin challenge, the pressure volume loops showed a distinct suppression of volume (FIG. 34A). When the lung volume was measured at a pressure of 30 cmH₂O (FIG. 34B), there was a significant reduction in volume that was partially restored by treatment with the PDE4-inhibitor of formula A′ (31%, P<0.05).

Computed tomography (CT) made use of computer-processed combinations of many X-ray images taken from different angles to produce cross-sectional (tomographic) images (virtual “slices”) of specific areas of a scanned object, allowing the user to see inside the object without cutting. This could be used to measure the volume of remodeled pulmonary tissue and is often expressed as a ratio of the total lung volume (to correct for differences in lung size between animals) Bleomycin treatment resulted in an increased ratio (FIG. 34C) that was partially reversed upon treatment with the PDE4-inhibitor of formula A′ (64%, P<0.05).

Osteopontin is known to modulate the recruitment and activation of inflammatory cells, such as macrophages and neutrophils, which plays a significant role in the development of lung fibrosis. Additionally, Osteopontin has been shown to promote tissue remodeling by influencing the migration, adhesion, and proliferation of various cell types, including fibroblasts and epithelial cells (Pardo A, Gibson K, Cisneros J, Richards T J, Yang Y, Becerril C, Yousem S, Herrera I, Ruiz V, Selman M, Kaminski N. Up-regulation and profibrotic role of osteopontin in human idiopathic pulmonary fibrosis. PLoS Med. 2005 September; 2(9):e251).

Measuring Osteopontin in the bleomycin rat model can provide valuable insights into the progression of the disease and the effectiveness of potential therapeutic interventions.

Osteopontin protein levels in lung tissue increased upon Bleomycin challenge from approx. 200 to approx. 550 pg/mL. Treatment with the PDE4-inhibitor of formula A′ reduced OPN levels in lung tissue by approx. 49%.

### 5. IN VITRO STUDY CONCERNING THE INHIBITORY EFFECT OF THE PDE4-INHIBITOR OF FORMULA A′ ON THE RELEASE OF DIFFERENT BIOMARKERS OF HUMAN SMALL AIRWAY EPITHELIAL CELLS (SAEC) WHICH WERE STIMULATED BY AN IPF-RELEVANT COCKTAIL (IPF-RC)

### 5.1 Summary

The PDE4-inhibitor of formula A′ inhibits the release of the biomarkers MMP7 (matrix metalloproteinase 7), PAI1 (plasminogen activator inhibitor 1), OPN (osteopontin), and CTGF (connective tissue growth factor) in IPF-rc-stimulated SAEC in a concentration dependent manner.

### 5.2 Method

Small airway epithelial cells of primary human origin at passage 4 were seeded in PneumaCult-Ex Plus complete medium onto rat tail collagen type I (30 μg/mL in PBS) coated 24-well plates.

After incubation for 24 hrs at 37° C. and 5% CO₂ cells were pre-stimulated with the PDE4-inhibitor of formula A′ for 30 min in different concentrations in PneumaCult-Ex Plus starving medium.

Afterwards an IPF-relevant cytokine cocktail (Schruf E, Schroeder V, Le H Q, Schönberger T, Raedel D, Stewart E L, Fundel-Clemens K, Bluhmki T, Weigle S, Schuler M, Thomas M J, Heilker R, Webster M J, Dass M, Frick M, Stierstorfer B, Quast K, Garnett J P. Recapitulating idiopathic pulmonary fibrosis related alveolar epithelial dysfunction in a human iPSC-derived air-liquid interface model. FASEB J. 2020 June; 34(6):7825-7846) was added to induce fibrotic changes. 72 hrs after stimulation supernatants were collected and analyzed using R&D DuoSet ELISAs following the manufacturers protocol.

### 5.3 Results

To determine the inhibitory potency of the PDE4-inhibitor of formula A′ on biomarker release of IPF-rc-stimulated primary human small airway epithelial cells, the supernatants of the cell culture from 5 different donors were analyzed.

FIG. 35 shows the concentration dependent inhibition of IPF-rc-induced release of MMP7 (A), sICAM-1 (B), OPN (C), and CTGF (D). The PDE4-inhibitor of formula A′ inhibited MMP7, sICAM, OPN, and CTGF with approx. IC₅₀-values of 4.4 μM, 537 nM, 1.1 μM, and 370 nM, respectively. The highest concentration of the PDE4-inhibitor of formula A′ (10 μM) led to a maximal inhibition of 63, 39, 67, and 75% for MMP7, sICAM, OPN, and CTGF, respectively (FIG. 35A-D).

The results of the biomarker analyses of the phase 2 trials is further summarized in the FIGURES.

### BRIEF DESCRIPTION OF THE FIGURES

FIG. 1: Adjusted mean (SE) for change from baseline in FVC hull over 12 weeks (MMRM) for the “non-AF-background” group (without background antifibrotics) for the “Compound of formula A′ treatment group” and for the “Placebo group”.

After 12 weeks of treatment the adjusted mean (SE) for change from baseline in FVC was +6.1 ml for the “Compound of formula A′ treatment group” and −95.6 ml for the “Placebo group” resulting Ma a difference of 101.7 ml.

FIG. 2: Adjusted mean (SE) for change from baseline in FVC hull over 12 weeks (MMRM) for the “AF-background” group (with background antifibrotics) for the “Compound of formula A′ treatment group” and for the “Placebo group”.

After 12 weeks of treatment the adjusted mean (SE) for change from baseline in FVC was +2.7 ml for the “Compound of formula A′ treatment group” and −77.7 ml for the “Placebo group” resulting Ma a difference of 80.4 ml.

FIG. 3: Adjusted mean (SE) for change from baseline in FVC hull over 12 weeks (MMRM) for the “pooled AF-background” group (group with background antifibrotics and group without background antifibrotics) for the “Compound of formula A′ treatment group” and for the “Placebo group”.

After 12 weeks of treatment the adjusted mean (SE) for change from baseline in FVC was +4.6 ml for the “Compound of formula A′ treatment group” and −83.8 ml for the “Placebo group” resulting Ma a difference of 88.4 ml.

FIG. 4: Mean change from baseline in FVC (ml) (95% confidence interval) at week 12 for the group without background antifibrotics (“non-AF-background group”) and for the group with background antifibrotics (“AF-background group”), with MMRM-analysis and with Bayesian borrowing.

FIG. 5: Adjusted mean (SE) for change from baseline in FVC hull at week 12 for the “pooled AF-background” group (group with background antifibrotics and group without background antifibrotics) for the “Compound of formula A′ treatment group” and for the “Placebo group”.

FIG. 6: Adjusted mean (95% confidence interval) for fold change from baseline (log 10) for KL-6 [U/ml] for the “Compound of formula A′ treatment group” and the “Placebo group”, both with “non-AF-background” (no antifibrotic background treatment).

The adjusted mean for fold change from baseline for KL-6 in the “Compound of formula A′ treatment group” is decreased compared to the “Placebo group” in patients with “non-AF-background”.

FIG. 7: Adjusted mean (95% confidence interval) for fold change from baseline (log 10) for KL-6 [U/ml] for the “Compound of formula A′ treatment group” and the “Placebo group”, both with “AF-background” (antifibrotic background treatment)

The adjusted mean for fold change from baseline for KL-6 in the “Compound of formula A′ treatment group” is decreased compared to the “Placebo group” in patients with “AF-background”.

FIG. 8: Adjusted mean (95% confidence interval) for fold change from baseline (log 10) for the Pulmonary surfactant protein SP-D [μg/L] for the “Compound of formula A′ treatment group” and the “Placebo group”, both with “non-AF-background” (no antifibrotic background treatment).

The adjusted mean for fold change from baseline for SP-D in the “Compound of formula A′ treatment group” is decreased compared to the “Placebo group” in patients with “non-AF-background”.

FIG. 9: Adjusted mean (95% confidence interval) for fold change from baseline (log 10) for the Pulmonary surfactant protein SP-D [μg/L] for the “Compound of formula A′ treatment group” and the “Placebo group”, both with “AF-background” (antifibrotic background treatment)

The adjusted mean for fold change from baseline for SP-D in the “Compound of formula A′ treatment group” is decreased compared to the “Placebo group” in patients with “AF-background”.

FIG. 10: Adjusted mean (95% confidence interval) for fold change from baseline (log 10) for CA-125 (also named MUC-16) [U/ml] for the “Compound of formula A′ treatment group” and the “Placebo group”, both with “non-AF-background” (no antifibrotic background treatment).

The adjusted mean for fold change from baseline for CA-125 in the “Compound of formula A′ treatment group” is decreased compared to the “Placebo group” in patients with “non-AF-background”.

FIG. 11: Adjusted mean (95% confidence interval) for fold change from baseline (log 10) for CA-125 (also named MUC-16) [U/ml] for the “Compound of formula A′ treatment group” and the “Placebo group”, both with “AF-background” (antifibrotic background treatment)

FIG. 12: Adjusted mean (95% confidence interval) for fold change from baseline (log 10) for the CA19.9 [U/ml] for the “Compound of formula A′ treatment group” and the “Placebo group”, both with “non-AF-background” (no antifibrotic background treatment).

FIG. 13: Adjusted mean (95% confidence interval) for fold change from baseline (log 10) for the CA19.9 [U/ml] for the “Compound of formula A′ treatment group” and the “Placebo group”, both with “AF-background” (antifibrotic background treatment)

FIG. 14: Adjusted mean (95% confidence interval) for fold change from baseline (log 10) for Matrilysin, also named MMP-7, [μg/L] for the “Compound of formula A′ treatment group” and the “Placebo group”, both with “non-AF-background” (no antifibrotic background treatment).

The adjusted mean for fold change from baseline for MMP-7 in the “Compound of formula A′ treatment group” is decreased compared to the “Placebo group” in patients with “non-AF-background”.

FIG. 15: Adjusted mean (95% confidence interval) for fold change from baseline (log 10) for Matrilysin, also named MMP-7, [μg/L] for the “Compound of formula A′ treatment group” and the “Placebo group”, both with “AF-background” (antifibrotic background treatment)

The adjusted mean for fold change from baseline for MMP-7 in the “Compound of formula A′ treatment group” is decreased compared to the “Placebo group” in patients with “AF-background”.

FIG. 16 Adjusted mean (95% confidence interval) for fold change from baseline (log 10) for Stromelysin, also named MMP-3, [μg/L] for the “Compound of formula A′ treatment group” and the “Placebo group”, both with “non-AF-background” (no antifibrotic background treatment).

FIG. 17: Adjusted mean (95% confidence interval) for fold change from baseline (log 10) for Stromelysin, also named MMP-3, [μg/L] for the “Compound of formula A′ treatment group” and the “Placebo group”, both with “AF-background” (antifibrotic background treatment)

FIG. 18: Adjusted mean (95% confidence interval) for fold change from baseline (log 10) for the Cartilage oligomeric matrix protein (COMP) [μg/L] for the “Compound of formula A′ treatment group” and the “Placebo group”, both with “non-AF-background” (no antifibrotic background treatment).

The adjusted mean for fold change from baseline for COMP in the “Compound of formula A′ treatment group” is decreased compared to the “Placebo group” in patients with “non-AF-background”.

FIG. 19: Adjusted mean (95% confidence interval) for fold change from baseline (log 10) for the Cartilage oligomeric matrix protein (COMP) [μg/L] for the “Compound of formula A′ treatment group” and the “Placebo group”, both with “AF-background” (antifibrotic background treatment)

FIG. 20: Adjusted mean (95% confidence interval) for fold change from baseline (log 10) for Prostasin [μg/L] for the “Compound of formula A′ treatment group” and the “Placebo group”, both with “non-AF-background” (no antifibrotic background treatment).

The adjusted mean for fold change from baseline for Prostasin in the “Compound of formula A′ treatment group” is decreased compared to the “Placebo group” in patients with “non-AF-background”.

FIG. 21: Adjusted mean (95% confidence interval) for fold change from baseline (log 10) for Prostasin [μg/L] for the “Compound of formula A′ treatment group” and the “Placebo group”, both with “AF-background” (antifibrotic background treatment)

FIG. 22: Adjusted mean (95% confidence interval) for fold change from baseline (log 10) for the von-Willebrand-Factor (vWF) [mg/L] for the “Compound of formula A′ treatment group” and the “Placebo group”, both with “non-AF-background” (no antifibrotic background treatment).

The adjusted mean for fold change from baseline for vWF in the “Compound of formula A′ treatment group” is decreased compared to the “Placebo group” in patients with “non-AF-background”.

FIG. 23: Adjusted mean (95% confidence interval) for fold change from baseline (log 10) for the von-Willebrand-Factor (vWF) [mg/L] for the “Compound of formula A′ treatment group” and the “Placebo group”, both with “AF-background” (antifibrotic background treatment)

FIG. 24: Adjusted mean (95% confidence interval) for fold change from baseline (log 10) for Soluable Intercellular adhesion molecule 1 (sICAM-1) [μg/L] for the “Compound of formula A′ treatment group” and the “Placebo group”, both with “non-AF-background” (no antifibrotic background treatment).

The adjusted mean for fold change from baseline for sICAM-1 is basically the same in the “Compound of formula A′ treatment group” and in the “Placebo group” in patients with “non-AF-background”. This speaks for the fact that sICAM-1 has at least no pharmacodynamic potential during IPF-treatment with the compound of formula A′.

FIG. 25: Adjusted mean (95% confidence interval) for fold change from baseline (log 10) for Soluable Intercellular adhesion molecule 1 (sICAM1) [μg/L] for the “Compound of formula A′ treatment group” and the “Placebo group”, both with “AF-background” (antifibrotic background treatment)

FIG. 26: Adjusted mean (95% confidence interval) for fold change from baseline (log 10) for the C-reactive protein (CRP) [mg/L] for the “Compound of formula A′ treatment group” and the “Placebo group”, both with “non-AF-background” (no antifibrotic background treatment).

The adjusted mean for fold change from baseline for CRP in the “Compound of formula A′ treatment group” is increased compared to the “Placebo group” in patients with “non-AF-background”.

FIG. 27: Adjusted mean (95% confidence interval) for fold change from baseline (log 10) for the C-reactive protein (CRP) [mg/L] for the “Compound of formula A′ treatment group” and the “Placebo group”, both with “AF-background” (antifibrotic background treatment)

The adjusted mean for fold change from baseline for CRP in the “Compound of formula A′ treatment group” is increased compared to the “Placebo group” in patients with “AF-background”.

FIG. 28: Adjusted mean (95% confidence interval) for fold change from baseline (log 10) for E-selectin [μg/L] for the “Compound of formula A′ treatment group” and the “Placebo group”, both with “non-AF-background” (no antifibrotic background treatment).

The adjusted mean for fold change from baseline for E-selectin in the “Compound of formula A′ treatment group” is decreased compared to the “Placebo group” in patients with “AF-background”.

FIG. 29: Adjusted mean (95% confidence interval) for fold change from baseline (log 10) for E-selectin [μg/L] for the “Compound of formula A′ treatment group” and the “Placebo group”, both with “AF-background” (antifibrotic background treatment)

The adjusted mean for fold change from baseline for E-selectin in the “Compound of formula A′ treatment group” is decreased compared to the “Placebo group” in patients with “AF-background”.

FIG. 30: Overview of Biomarker analysis with regard to the “pharmacodynamic potential” of the individual biomarkers during treatment with the compound of formula A′: In particular KL-6, SP-D (shown as PSP-D) and MMP7 (shown as Matrilysin) are decreased during the treatment with the PDE4-inhibitor of formula A′ and therefore show a pharmacodymanic potential, whereas C-reactive protein (CRP) is increased during the treatment with the PDE4-inhibitor of formula A′. sICAM-1, E-selectin, COMP and CA19-9 also seem to be decreased during the treatment with the PDE4-inhibitor of formula A′—however in a lower extent than KL-6, SP-D and MMP7 and predominently in the non-AF-groups.

FIG. 31: Overview of Biomarker analysis with regard to the “prognostic potential” of the individual biomarkers in IPF patients.

Baseline levels of KL-6, SP-D, CA-125 (=Mucin-16), sICAM-1 and in a slightly lower extent CA19-9—seem to be negatively associated with a decrease-from-baseline in lung function, measured as change-from-baseline (CfB) in FVC and as change-from-baseline (CfB) in Dlco % pred. at week 12, in IPF patients of the Placebo-groups only.

KL-6, SP-D, CA-125 and sICAM-1 (and CA19-9 in a slightly lower extent) therefore seem to have a prognostic potential in IPF patients.

FIG. 32: Analysis which of the tested biomarkers are “outcome-related” during treatment with the compound of formula A′.

The change in levels of biomarkers KL-6, SP-D, E-selectin and sICAM-1 during the treatment of IPF patients with the PDE4-inhibitor of formula A′ shows an association with the Change from Baseline (CfB) of the Forced Vital Capacity (FVC) and with the Change from Baseline (CfB) in the Diffusing capacity or Transfer factor of the lung carbon monoxide (Dlco) at week 12, in both, the “non-AF group” and the “AF group” of the trial. Therefore KL-6, SP-D, E-selectin and sICAM-1 seem to have an“outcome-related potential” during the treatment with the PDE4-inhibitor of formula A′.

FIG. 33: Summary of the “pharmacodynamic potential”, the “prognostic potential” and the “outcome-relation” of the tested biomarkers during treatment with the compound of formula A′.

FIG. 34: Treatment with the PDE4-inhibitor of formula A′ improved lung function in a therapeutic rat model of bleomycin-induced lung fibrosis (see A, B and C) and lead to a reduced expression of the osteopontin (OPN) protein in lung tissue (see D)

FIG. 35: After stimulation with an IPF-relevant cytokine cocktail the treatment with the PDE4-inhibitor of formula A′ inhibited the expression of IPF-associated proteins such as MMP-7 (FIG. 35A), sICAM-1 (FIG. 35B), OPN (FIG. 35 C) and CTGF (FIG. 35D) in human small airway epithelial cells.

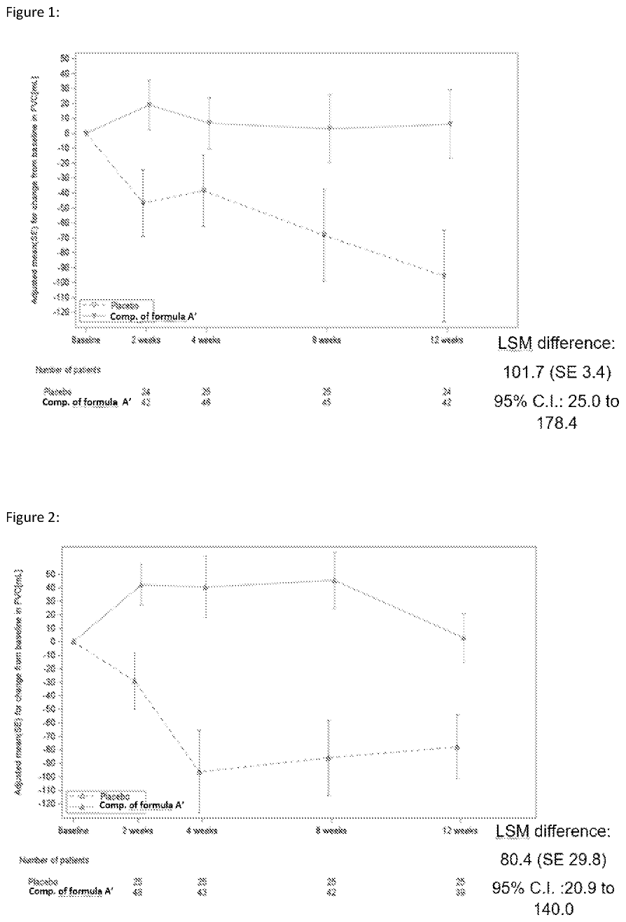

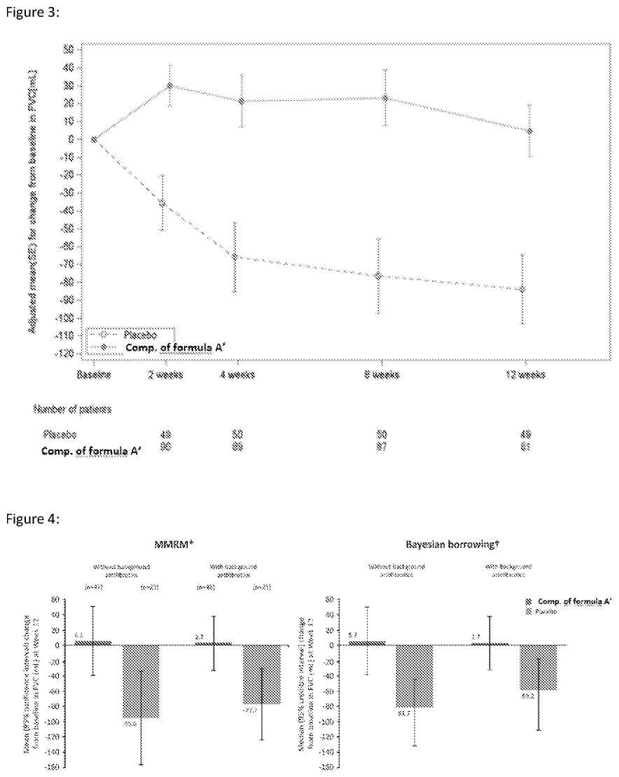

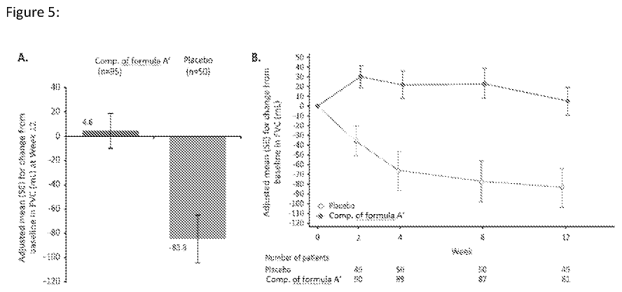

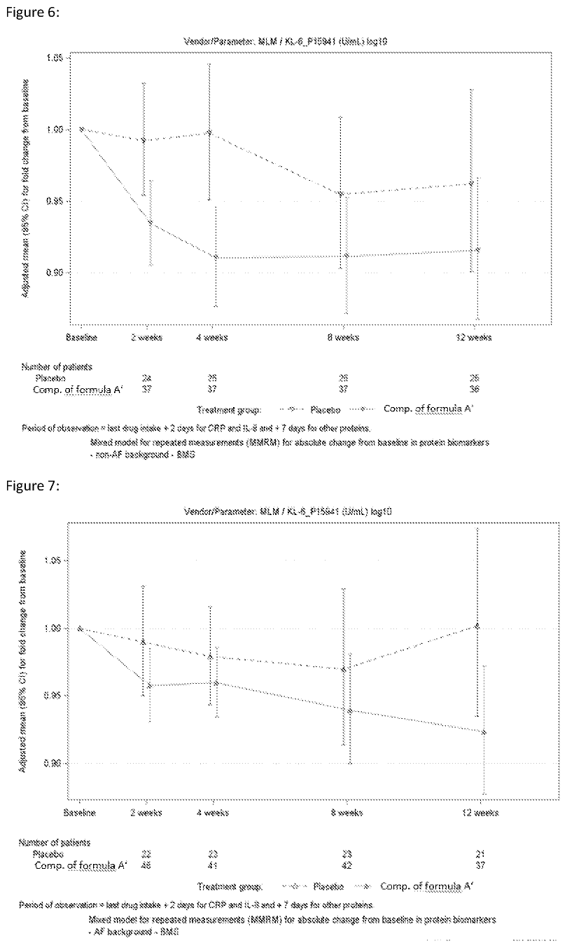

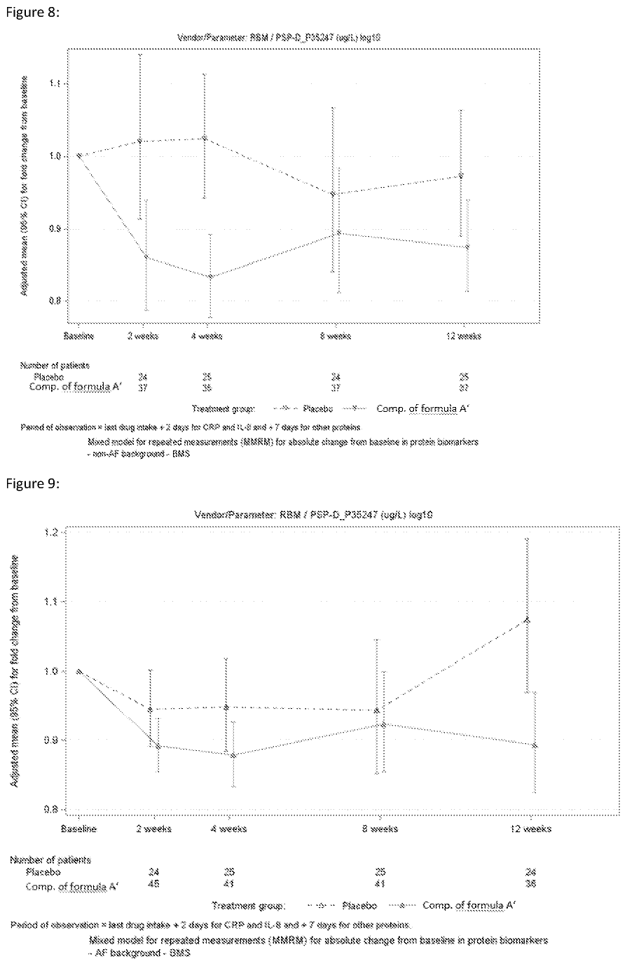

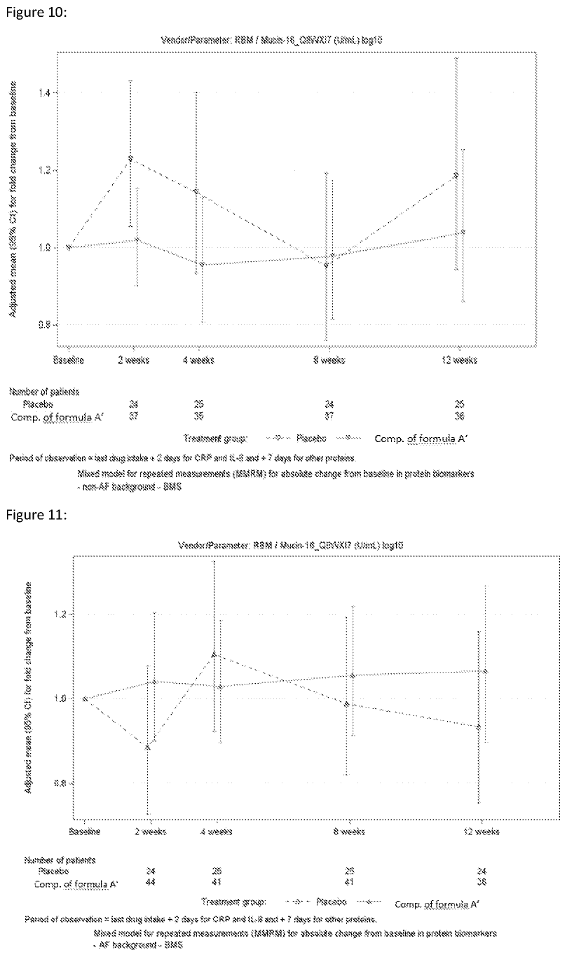

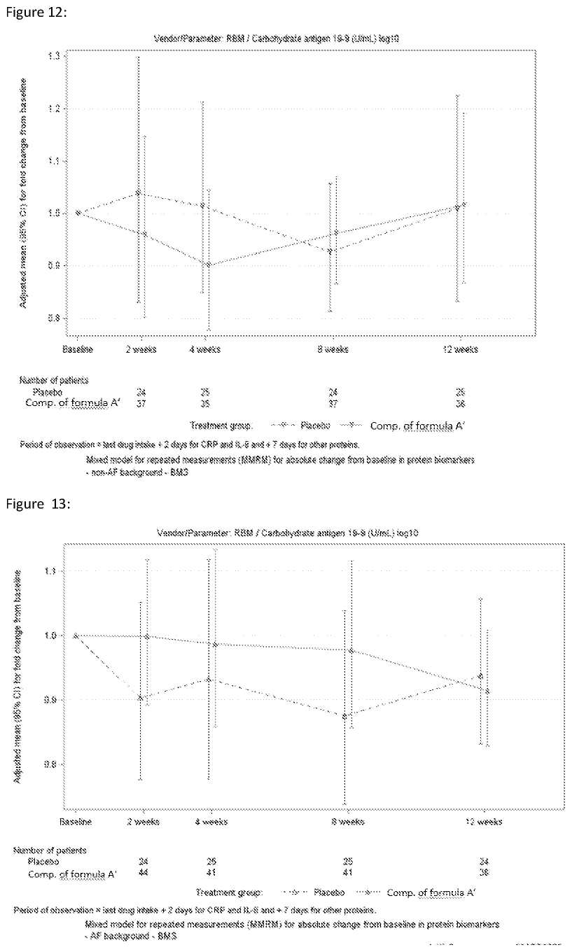

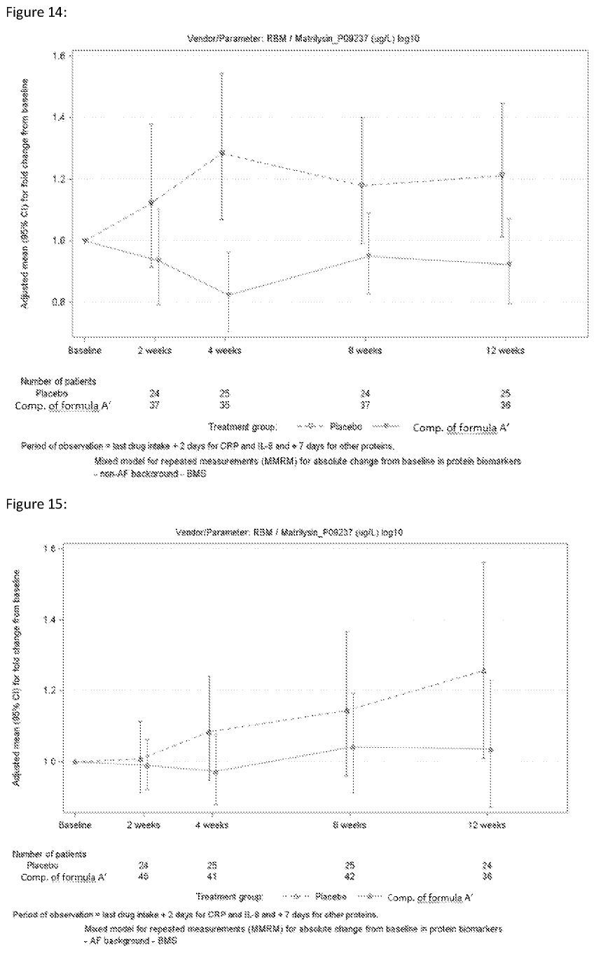

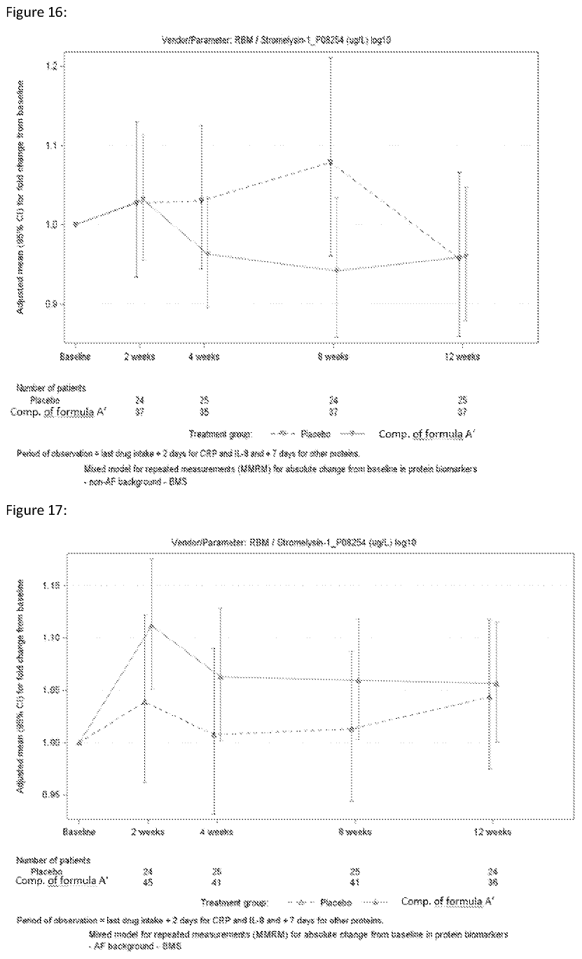

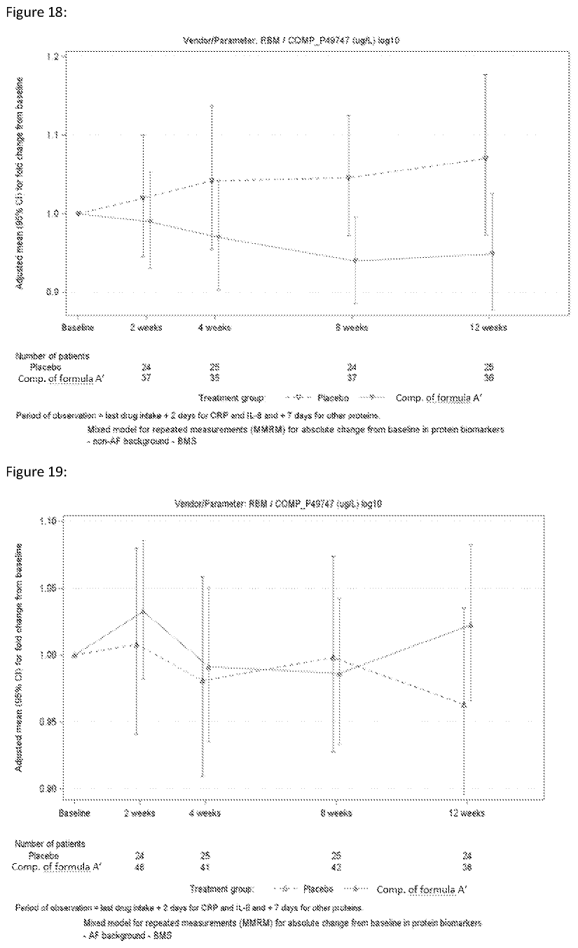

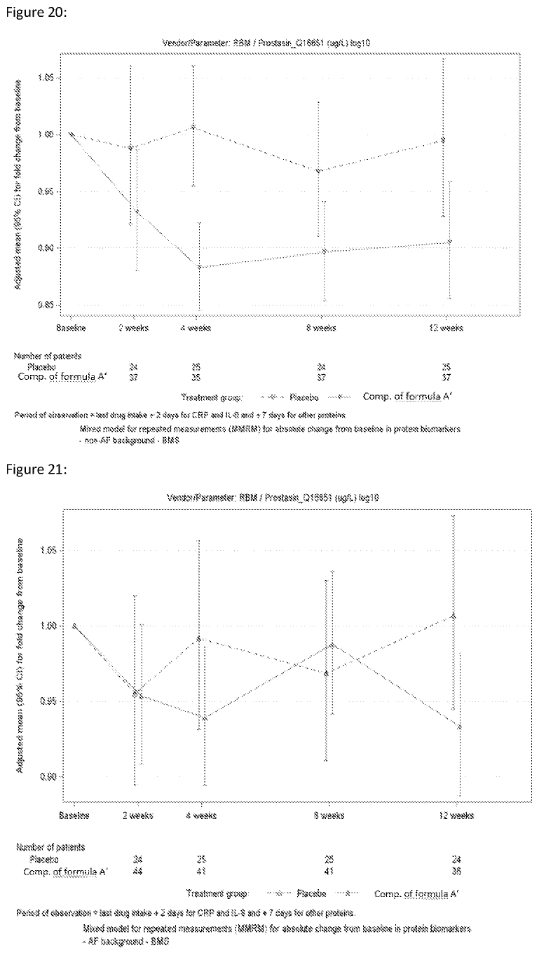

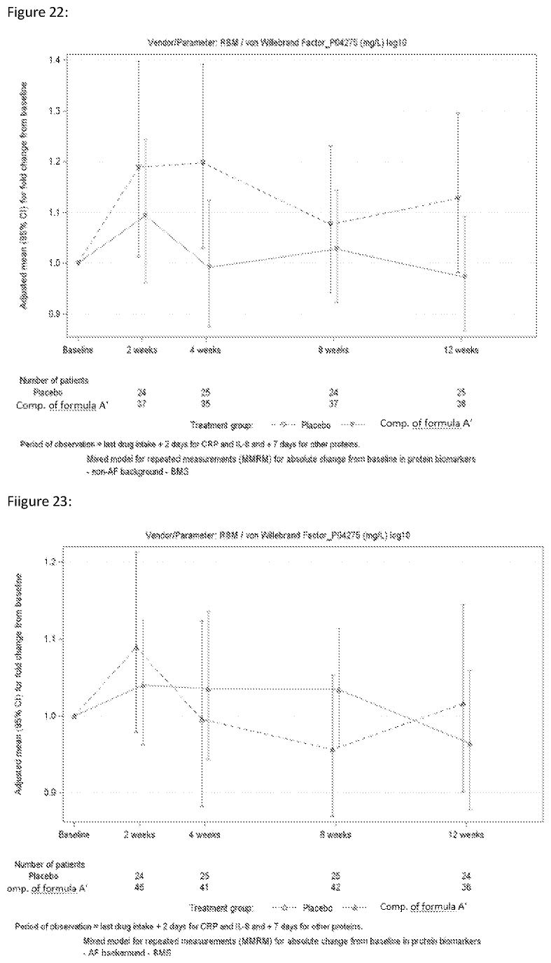

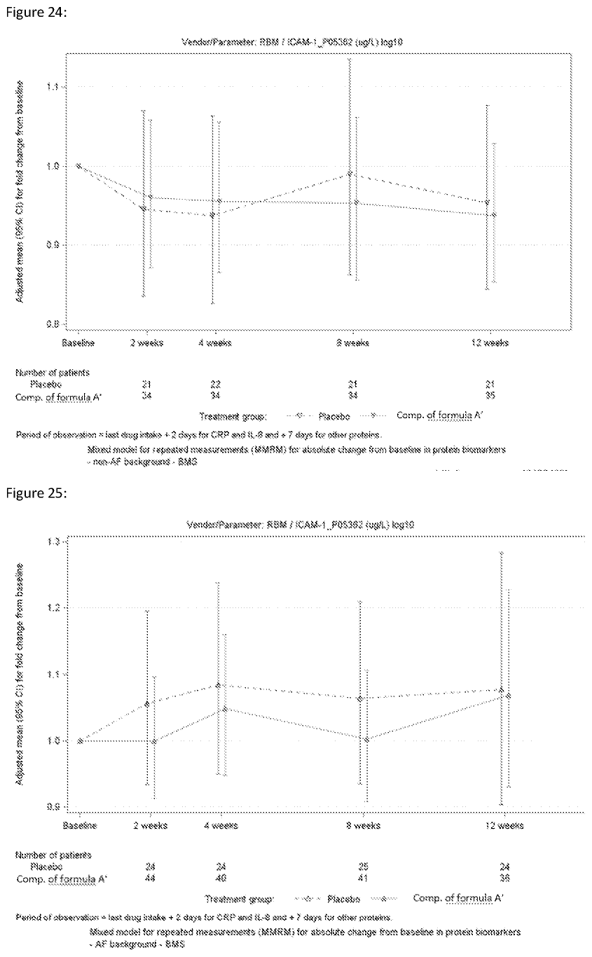

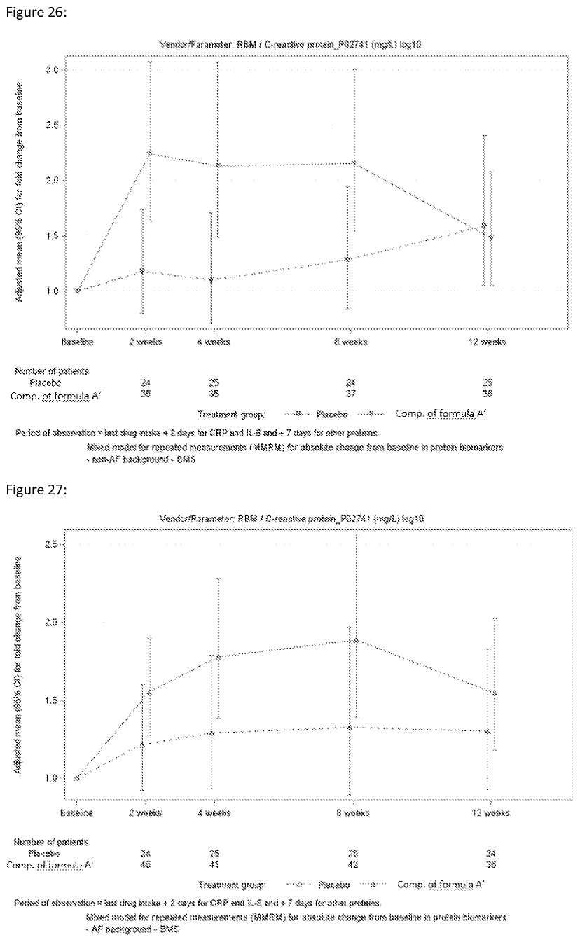

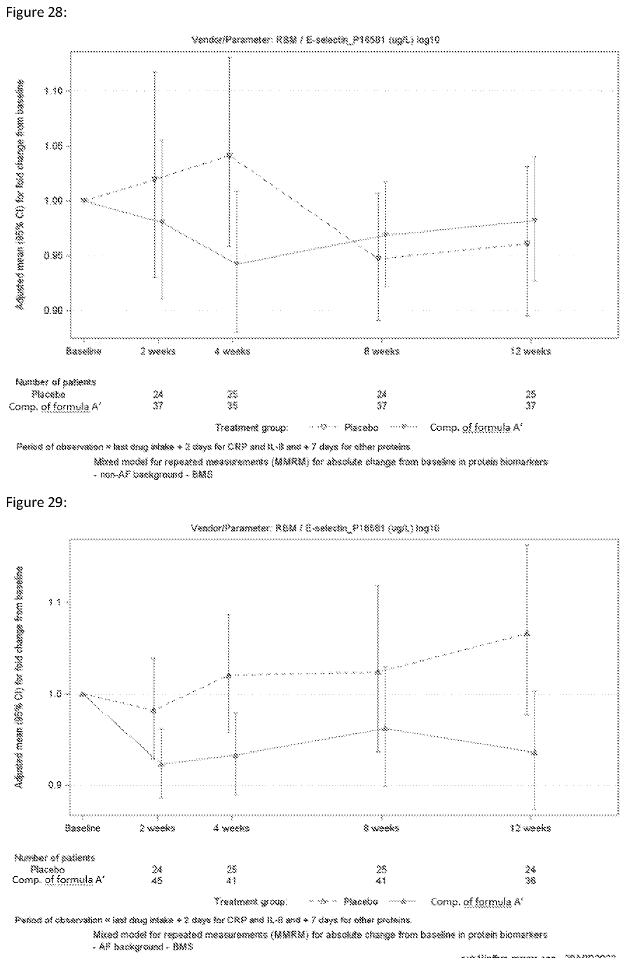

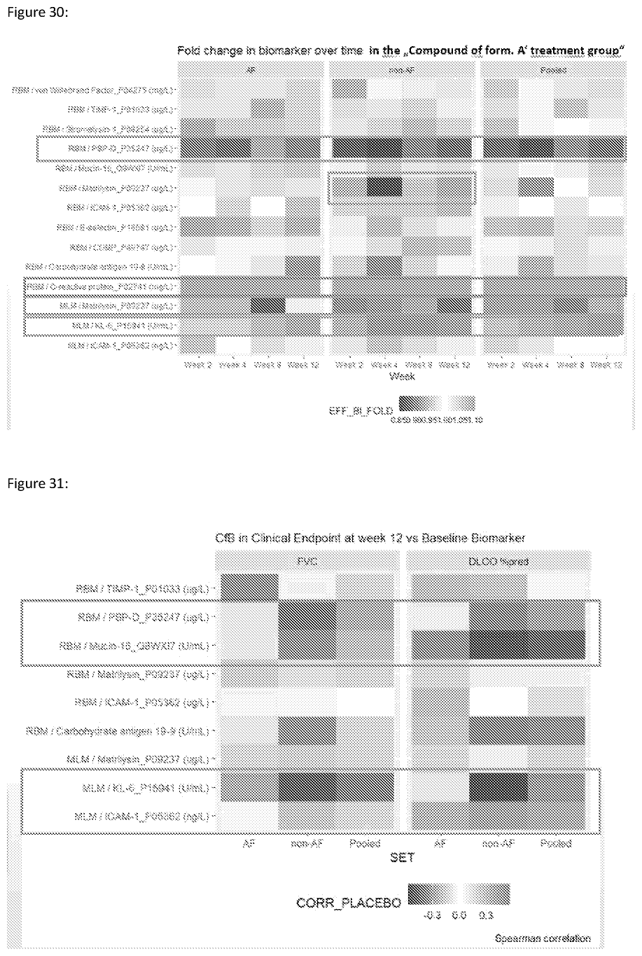

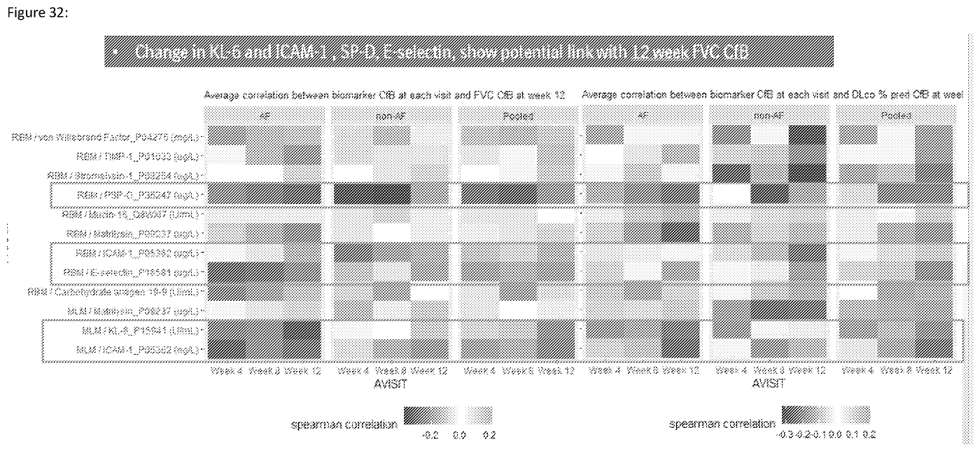

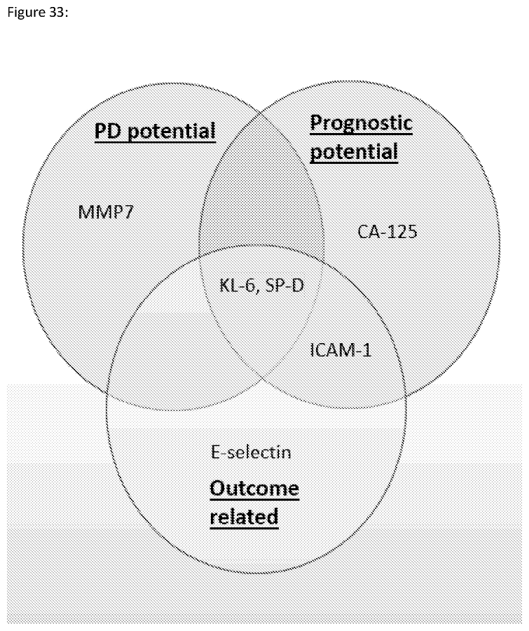

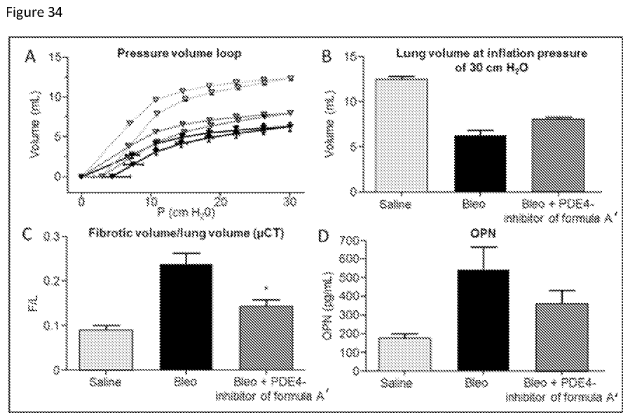

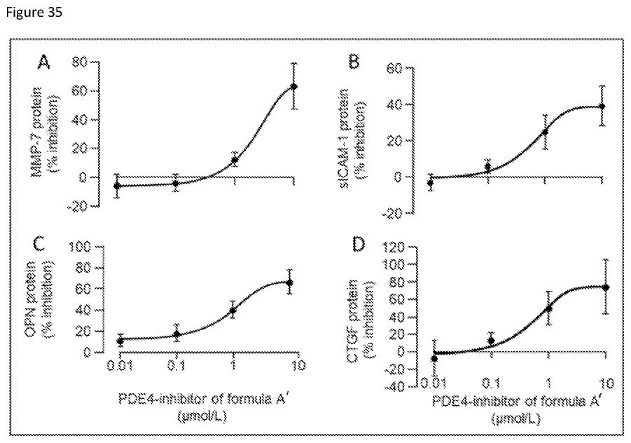

1. A method for the treatment of a progressive fibrosing interstitial lung disease (PF-ILD) in a patient comprising the following steps:
a) measuring or having measured the concentration, expression level or activity of one or more biomarkers selected from the group consisting of Krebs von den Lungen protein (KL-6), Pulmonary Surfactant Protein D (SP-D), Matrilysin (MMP7), CA-125 (also named MUC-16), E-selectin, Stromelysin (MMP3), CA-19-9, osteopontin (OPN), connective tissue growth factor (CTGF), Cartilage oligomeric matrix protein (COMP), prostasin, von-Willebrand-Faktor (vWF), soluble Intercellular adhesion molecule 1 (sICAM1) and C-reactive protein (CRP) in the serum or plasma of a blood sample obtained from said patient,
b) comparing or having compared the concentration, expression level or activity of the one or more biomarkers as listed in step a) in said patient's blood serum or blood plasma sample to a reference concentration, expression level or activity of the respective one or more biomarkers,
c) determining or having determined that the concentration, expression level or activity of the respective one or more biomarkers as listed in step a) is modified compared to a respective reference concentration, expression or activity of that respective one or more biomarker, and
d) administering to said patient a therapeutically efficient amount of the PDE4-inhibitor of the following formula A′:

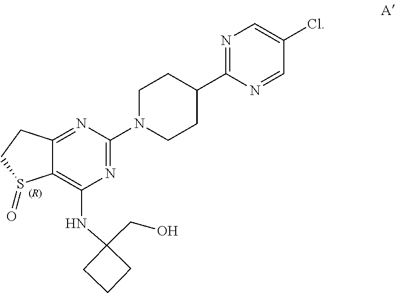

2. The method according to claim 1, wherein the one or more biomarkers of step a) is selected from the group consisting of KL-6, SP-D, MMP7, CA-125, E-selectin, sICAM-1, MMP3, CA19-9, OPN, CTGF, COMP, prostasin and vWF and wherein step c) comprises determining or having determined that the concentration, expression level or activity of the respective one or more biomarkers selected from the group consisting of KL-6, SP-D, MMP7, CA-125, E-selectin, sICAM-1, MMP3, CA19-9, OPN, CTGF, COMP, prostasin and vWF is increased compared to a respective reference concentration, expression or activity of that respective one or more biomarker.

3. The method according to claim 1, wherein the one or more biomarkers of step a) is C-reactive protein (CRP) and wherein step c) comprises determining or having determined that the concentration of CRP is decreased compared to a respective reference concentration of CRP.

4. The method according to claim 1, wherein the progressive fibrosing interstitial lung disease (PF-ILD) is idiopathic pulmonary fibrosis (IPF).

5. The method according to claim 1, wherein the patient has already been treated by an antifibrotic compound selected from the group consisting of nintedanib, pirfenidone and any pharmaceutically acceptable salts thereof prior to steps a), b), c) and d).

6. The method according to claim 5, wherein the treatment with the antifibrotic compound selected from the group consisting of nintedanib, pirfenidone and any pharmaceutically acceptable salts thereof prior to steps a), b), c) and d) is continued as background treatment during the treatment with the compound of formula A′ in step d).

7. The method according to claim 5, wherein the treatment with the antifibrotic compound selected from the group consisting of nintedanib, pirfenidone and any pharmaceutically acceptable salts thereof prior to steps a), b), c) and d) is not continued as background treatment during the treatment with the compound of formula A′ in step d).

8. The method according to claim 1, wherein 18 mg of the PDE4-inhibitor of formula A′ is administered twice daily to the patient in step d).

9. The method according to claim 1, wherein the concentration, expression level or activity of the respective one or more biomarker in step c) is a concentration, expression level or activity of the respective one or more biomarker which is larger than the reference concentration and which is considered as prognostic for PF-ILD progression.

10. The method according to claim 1, wherein the concentration, expression level or activity of the respective one or more biomarker in step c) is a concentration, expression level or activity of the respective one or more biomarker which is larger than the reference concentration and which is considered as prognostic for IPF progression.

11. The method according to claim 1, wherein the one or more biomarkers of step a) are selected from the group consisting of Krebs von den Lungen protein (KL-6), Pulmonary Surfactant Protein D (SP-D), Matrilysin (MMP7), CA-125 (also named MUC-16), CA19-9, OPN, soluble Intercellular adhesion molecule 1 (sICAM-1) and E-selectin and wherein step c) comprises determining or having determined that the concentration, expression level or activity of these respective one or more biomarkers selected from the group consisting of KL-6, SP-D, MMP7, CA-125, CA19-9, OPN, sICAM-1 and E-selectin is increased compared to a respective reference concentration, expression or activity of that respective one or more biomarker.

12. The method according to claim 1, wherein the one or more biomarkers of step a) are selected from the group consisting of Krebs von den Lungen protein (KL-6), Pulmonary Surfactant Protein D (SP-D), Matrilysin (MMP7) and E-selectin and wherein step c) comprises determining or having determined that the concentration, expression level or activity of these respective one or more biomarkers selected from the group consisting of KL-6, SP-D, MMP7 and E-selectin is increased compared to a respective reference concentration, expression or activity of that respective one or more biomarker.

13. The method according to claim 1, wherein the one or more biomarkers of step a) are selected from the group consisting of Krebs von den Lungen protein (KL-6), Pulmonary Surfactant Protein D (SP-D) and Matrilysin (MMP7) and wherein step c) comprises determining or having determined that the concentration, expression level or activity of these respective one or more biomarkers selected from the group consisting of KL-6, SP-D and MMP7 is increased compared to a respective reference concentration, expression or activity of that respective one or more biomarker.

14. The method according to claim 1, wherein the one or more biomarkers of step a) are selected from the group consisting of Krebs von den Lungen protein (KL-6) and Pulmonary Surfactant Protein D (SP-D) and wherein step c) comprises determining or having determined that the concentration, expression level or activity of these respective one or more biomarkers selected from the group consisting of KL-6 and SP-D is increased compared to a respective reference concentration, expression or activity of that respective one or more biomarker.

15. The method according to claim 1, wherein the one or more biomarkers of step a) is Krebs von den Lungen protein (KL-6) and wherein step c) comprises determining or having determined that the concentration, expression level or activity of KL-6 is increased compared to a respective reference concentration, expression or activity of KL-6.

16. The method according to claim 1, wherein the one or more biomarkers of step a) is SP-D and wherein step c) comprises determining or having determined that the concentration, expression level or activity of SP-D is increased compared to a respective reference concentration, expression or activity of SP-D.
